(19) United States
(12) Patent Application Publication
(10) Pub. No.: US 2022/0267308 A1
(43) Pub. Date: Aug. 25, 2022

(54) Modulators of Excitatory Amino Acid Transporters and Methods Using Same

(71) Applicants: The Wistar Institute of Anatomy and Biology, Philadelphia, PA (US); Drexel University, Philadelphia, PA (US)

(72) Inventors: Joseph M. Salvino, Philadelphia, PA (US); Andreia C.K. Mortensen, Philadelphia, PA (US); Valli Venkata Srikanth Yellamelli, Philadelphia, PA (US)

(21) Appl. No.: 17/628,106

(22) PCT Filed: Jul. 17, 2020
(86) PCT No.: PCT/US2020/042543

§ 371 Date: Jan. 18, 2022

Related U.S. Application Data
(60) Provisional application No. 62/875,631, filed on Jul. 18, 2019.

(51) Int. Cl. C07D 403/06; C07D 401/14; C07D 241/04; C07D 401/06; A61P 25/28

(52) U.S. Cl. CPC C07D 403/06; C07D 401/14; A61P 25/28; C07D 401/06; C07D 241/04

(57) ABSTRACT

The present disclosure provides in one aspect compounds of Formula I. In certain embodiments, the compounds of the disclosure are useful for treating, ameliorating or preventing a disease or disorder that is caused, induced or characterized by abnormal reduction in glutamate transporter activity or abnormal increase in extracellular CNS glutamate concentration in a subject. In certain embodiments, the compounds of the disclosure stimulate a glutamate transporter.

### CROSS-REFERENCE TO RELATED APPLICATIONS

The present application claims priority under 35 U.S.C. § 119(e) to U.S. Provisional Patent Application No. 62/875,631, filed Jul. 18, 2019, the disclosure of which is incorporated herein by reference in its entirety.

### BACKGROUND OF THE INVENTION

Glutamate is the predominant excitatory amino acid neurotransmitter in the mammalian central nervous system (CNS) and is essential for normal brain function, including cognition, memory, learning, developmental plasticity, and long-term potentiation.

The termination of glutamate neurotransmission is achieved by rapid uptake of the released glutamate by presynaptic and astrocytic sodium-dependent transporters. There are five subtypes of excitatory amino-acid transporters or glutamate transporters: EAAT1 or GLAST, EAAT2 or GLT-1, EAAT3 or EAAC1, EAAT4, and EAAT5. The major regulator of extracellular glutamate levels in the brain is EAAT2, which is expressed in astroglial cells and responsible for about 90% total glutamate uptake in the CNS and about 1% of total brain protein in the CNS.

Glutamate translocation is a multi-step process that uses energy derived from Na⁺ and K⁺ electrochemical gradients to move glutamate against its concentration gradient. The rate-limiting step in the cycle involves translocation of K⁺ and reorientation of the carrier to become accessible for a new substrate molecule in the synaptic cleft. Despite the identification of several crystal structures of a bacterial homolog of glutamate transporters Glt(ph), and the identification of the crystal structure of the human EAAT1 transporter, no rationally designed transporter activator has been identified so far.

Excitotoxicity plays a key role in secondary damage following acute pathologies, such as traumatic brain injury (TBI), hyper-excitability and seizures, stroke, epilepsy, cerebral and retinal ischemia; and chronic pathologies, such as Amyotrophic lateral sclerosis (ALS), Alzheimer's disease, Huntington's disease, HIV-associated neurocognitive disorder (HAND), neuropathic pain and drug abuse/addiction.

Ischemic events in humans and in animals lead to an acute and sustained increase in extracellular glutamate concentrations, indicative of a lack of timely clearance by glutamate transporters. In fact, dysfunctional glutamate transporters are often the initiating event or part of the cascade leading to brain injury. Reductions in EAAT2 activity result in increased predisposition for seizures and susceptibility to damage due to ischemia. Excessive activation of glutamate receptors due to sustained elevation of extracellular glutamate levels results in Ca²⁺ influx and activation of a cascade of phospholipases, endonucleases and proteases such as calpain that can lead to apoptotic or necrotic cell death. In addition, ischemic events in humans and animals lead to an acute and sustained increase in extracellular glutamate concentrations, which suggests a lack of proper clearance by glutamate transporters. In fact, dysfunctional glutamate transporters are often the initiating event or part of the cascade leading to brain injury.

Knockout of the EAAT2 gene resulted in exacerbated damage compared to their wild-type counterparts following cerebral injury in mice and controlled cortical impact in rats. Additionally, a transgenic approach for EAAT2 overexpression with double transgenic mice created from crossing an ALS mouse model to a mouse model overexpressing EAAT2 resulted in animals that display delayed grip strength decline, motor neuron loss, and increased life expectancy.

This class of compounds could also be developed to treat drug addiction and relapse. Over the past 20 years the addiction field has ascribed a critical role for glutamatergic transmission in the development of addiction. Chronic drug use produces enduring neuroadaptations in corticostriatal projections that are believed to contribute to a maladaptive deficit in inhibitory control over behavior. Much of this research focuses on the role played by ionotropic glutamate receptors directly involved in long-term potentiation and depression or metabotropic receptors indirectly modulating synaptic plasticity. Importantly, the balance between glutamate release and clearance tightly regulates the patterned activation of these glutamate receptors, emphasizing an important role for glutamate transporters in maintaining extracellular glutamate levels. Recent evidence suggests that glutamate transporters can be modulated by chronic drug use at a variety of levels. Preclinical and clinical data suggests that glutamate transporters offer an effective target for the treatment of drug addiction.

Several classes of compounds that target stages in these processes, such as NMDA receptor antagonists and calcium influx inhibitors, alleviate cellular damage and neurologic deficits to some extent, but have limited clinical use due to substantial side effects. Transcriptional or translational upregulators of EAAT2, such as GPI-1046, ceftriaxone, harmine and pyridazine derivatives, are neuroprotective through selective augmentation of EAAT2 expression. Nevertheless, these compounds must be administered prophylactically to be neuroprotective, and thus they have low clinical relevance for acute conditions. Some compounds with neuroprotective properties, such as MS-153, riluzole, guanosine and nicergoline, acutely stimulate glutamate uptake by an indirect modulation of transporter activity, but are non-specific and cause numerous side effects.

Currently there is a critical lack of compounds that activate, stimulate and/or upregulate the activity of glutamate transporters, such as but not limited to EAAT2. Such compounds should be useful for treating diseases or disorders that are caused, induced, or characterized by abnormal reduction in glutamate transporter activity or abnormal increase in extracellular CNS glutamate concentration in a subject. The present disclosure fulfills this need.

### BRIEF SUMMARY OF THE DISCLOSURE

The present disclosure provides certain compounds of Formula I or II, or an enantiomer, diastereoisomer, tautomer, salt or solvate thereof, having the formula:

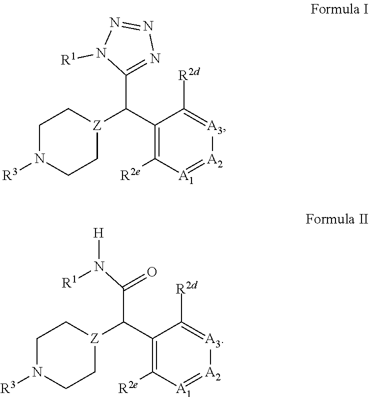

wherein R¹, R^{2d}, R^{2e}, R³, Z, and A¹-A³ are defined elsewhere herein.

The present disclosure further provides methods of treating, preventing, and/or ameliorating a disease or disorder that is caused, induced or characterized by abnormal reduction in glutamate transporter activity or abnormal increase in extracellular CNS glutamate concentration in a subject, the method comprising administering to the subject a therapeutically effective amount of a compound contemplated within the disclosure.

### BRIEF DESCRIPTION OF THE FIGURES

The drawings illustrate generally, by way of example, but not by way of limitation, various embodiments of the present application.

FIGS. 1A-1B show the effect of compound DA-023 on glutamate uptake mediated by EAAT2-expressing COS-7 cells. FIG. 1A shows dose response curves illustrating potency in the nM range and efficacy of <150% on transport augmentation. FIG. 1B illustrates a kinetic analysis for DA-023 (10 nM) in presence of different concentrations of glutamate, showing increase in Vₘₐₓ but no effect on K_{M} (affinity for the substrate).

FIGS. 2A-2F show the neuroprotective effects of GT949 and DA-023 in neuronal cultures in the bi-laminar approach. Top: Representative images (40× magnification) of 14 DIV cortical neurons in control conditions (FIG. 2A) or subjected to insult with 10 μM L-glutamate for 24 h (FIG. 2B). FIG. 2C shows quantification of administration of 10 nM GT949, 10 nM DA-023 and 100 μM APV attenuate cell death. Bottom: Representative images of cortical neurons in control conditions (FIG. 2D) or subjected to insult with OGD for 20 min (FIG. 2E). FIG. 2F shows quantification of administration of 100 nM DA-023 and 100 μM APV attenuated cell death, whereas negative control DA-051 did not. Quantification performed 24 h after treatments in cultures stained for MAP-2 (green) and DAPI (blue). Surviving neurons were estimated by blind counting of 10 fields/coverslips, averages of triplicate determinations±SD (ANOVA followed by Dunnet's posthoc test, *p<0.05, **<0.01, p***<0.001, compared to control, #p<0.05, ###p<0.001, compared to insult).

FIG. 3 shows the in vivo dose response effect of DA-023 in the SNI neuropathic pain model. Rats (n=5/group) were subjected to ligation of two of the three branches of the sciatic nerve. Seven days later, the pain response was determined by testing with von Frey filaments of increasing bending force. DA-023 was administered (IP) 30 minutes prior to testing (*p<0.05, compared to control).

FIGS. 4A-4C show the effects of NA-014 on glutamate transporters. FIG. 4A shows the effect of compound NA-014 on glutamate uptake mediated by EAAT1-3-expressing COS-7 cells, showing selective effect for EAAT2. FIG. 4B shows a dose response curve showing potency in the nM range and efficacy of <150% on transport augmentation. Effects of NA-014 on the kinetics of EAAT2 in transfected COS-7 cells. Graphs illustrate kinetic analysis of L-glutamate uptake in transfected COS-7 cells pre-incubated with 10, 100 and 500 nM of NA-014. K_{M} was not statistically different. Vₘₐₓ at 10 nM concentration was not statistically different from that of vehicle. *** p<0.05 Vmax of compound compared (100 nM, 500 nM) to vehicle. FIG. 4C shows the effect of NA-014 on the kinetics of glutamate transport in glia cells. Graphs illustrate kinetic analysis of L-glutamate uptake in cultured glia cells pre-incubated with vehicle, 100 nM, and 500 nM of NA-014. K_{M} was not statistically different. *** p<0.05 V of drug compared to vehicle.

FIGS. 5A-5D show the neuroprotective effects of NA-014 in neuronal cultures in the mixed neuron-glia approach. Representative images (30×) from 14-DIV neuron-glia cultures (FIG. 5A) Vehicle (control); (FIG. 5B) Neuron-glia cultures treated with 100 M L-glutamate for 24 h; (FIG. 5C) Co-treatment with 100 μM L-glutamate and 100 nM NA-014; (FIG. 5D) quantification showing the neuroprotective effect of NA-014, which is abolished by co-application of EAAT inhibitor TBOA (threo-β-benzyloxyaspartate), suggesting its neuroprotective effects are dependent on the transport. Immunostaining against neuronal marker MAP-2 (green), glial maker GFAP (red), and nuclear marker DAPI (blue). Surviving neurons were estimated by blind counting of 10 fields/coverslips, averages of triplicate determinations±SD (ANOVA followed by Dunnet's posthoc test, ***p<0.001, compared to control, ###p<0.001, compared to insult).

FIG. 6 shows an example synthetic scheme for the synthesis of compound DA-023 using the Azido-Ugi multi-component reaction.

FIGS. 7A-7E show the in vitro neuroprotective properties of second and third generation compounds after glutamate and oxygen glucose deprivation insults in mixed neuron/glia cultures. FIG. 7A shows representative images from 14-DIV neuron/glia cultures, obtained from prefrontal cortices from late embryonic stage (E17) rat embryos and plated at a density of 35,000 cells/coverslip in 12-well plates. Vehicle (control), cells treated with application of 100 μM L-glutamate for 24 h and with co-treatment with 100 nM NA-014 are indicated. Immunostaining against neuronal marker MAP-2 (green), glial maker GFAP (red), and nuclear marker DAPI (blue). Images taken with 30× magnification with a confocal microscope. FIG. 7B-7E shows the quantification of neuronal survival after L-glutamate insult (B and C) and OGD insult (D and E) and the effect of compounds NA-005 and NA-014 and positive control AP-V.

FIGS. 8A-8F shows the effect of DA-023 on neurodegeneration and axonal injury following lateral fluid-percussion brain injury. Representative photomicrographs of FJ-B-(Fluorojade-B, a marker of neurodegeneration) stained sections from the cortex (FIGS. 8A-8C) and area CA3 of the hippocampus (FIGS. 8D-8F). Sham-injured (FIG. 8A and FIG. 8D) animals received the higher dose of compound DA-023 (8.25 mg/Kg) while brain-injured animals were vehicle-treated (FIG. 8B, FIG. 8E) or treated 8.25 mg/Kg of DA-023 (FIG. 8C and FIG. 8F). Note the absence of FJ-B (+) profiles in sham-injured animals (FIG. 8A and FIG. 8D). All images taken at 20× magnification.

FIGS. 9A-9E shows the quantitative analyses of the experiments in FIG. 9 in graphs (FIG. 9A) for cortex analysis and (FIG. 9B) for hippocampus. Values are presented as means and standard deviation of Fluorojade-B (+) cells/HPF. (FIGS. 9C-9D): Representative photomicrographs demonstrating intra-axonal immunoreactivity for β-amyloid precursor protein (β-APP), comparing animals that received saline (FIG. 9C) and the 3.75 mg/Kg of DA-023 (FIG. 9D). FIG. 9E: quantitative analysis of APP staining in the thalamus and white matter combined.

FIGS. 10A-10D shows the effect of DA-023 in the pilocarpine in vivo model of epilepsy. Rats were administered 380 mg/Kg pilocarpine, followed by administration of Thiopental (30 mg/Kg) and the implantation of an osmotic pump to deliver 8 μl/h/48 hours of DA-023 to the final dose of 4 mg/Kg. Following this period animals were perfused and brains subjected to histological analysis. Representative images stained with Fluorojade C(+) (marker of neurodegeneration) of CA1, CA3, hylus and dentate gyrus regions of the hippocampus in coronal sections (20 μm) of right (FIG. 10A) and left (FIG. 10B) side of brains from animals subjected to Status Epilepticus (SE) and administered saline or DA-023, at 40× magnification. FIG. 10C shows quantifications of FJC+ cell numbers in the CA1, CA3; FIG. 10D shows effects of administration of saline or DA-023 in hilus and gyrus dentate. A significant reduction in the neuronal death was found in the hilus (F(2.22)=4.780; p=0.0189; DF=2).

FIGS. 11A-11C shows the effects of DA-023 and NA-014 in neuropathic pain. In vivo results in the spare nerve injury (SNI) pain model. FIG. 11A illustrates a nerve model (image from Duraku et al. Mol Pain; 8:61, 2012). In vivo dose response effect of DA-023 (FIG. 11B) and NA-014 (FIG. 11C) in the SNI neuropathic pain model. Rats (n=5/group) were subjected to ligation of two of the three branches of the sciatic nerve. 7 days later, the pain response was determined by testing with von Frey filaments of increasing bending force. DA-023 or NA-014 were administered (IP) 30 minutes prior to testing (*p<0.05, **p<0.01 and ***p<0.001, compared to control).

FIG. 12 shows the in vivo evaluation of NA-014 in the SNL model performed at Melior Discovery, Inc. NA-014 at the 10 mg/kg dose and Gabapentin (200 mg/kg) showed an analgesic response (**p<0.01, compared to vehicle).

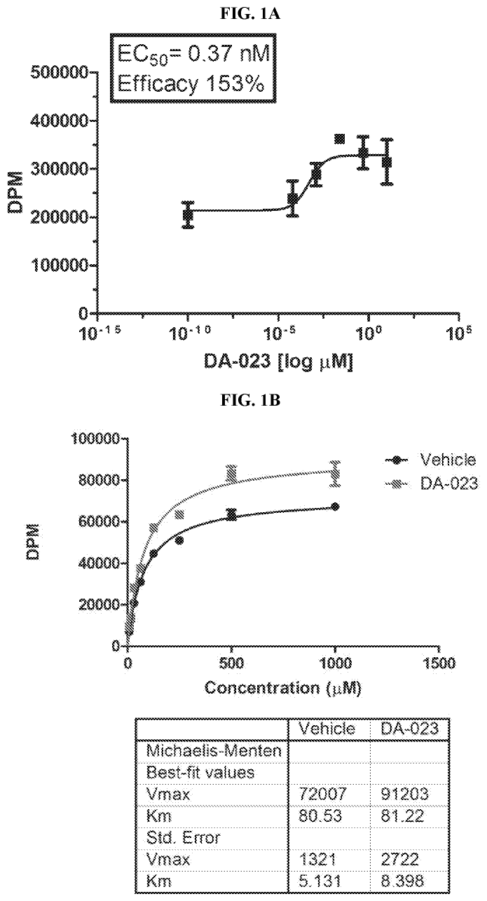

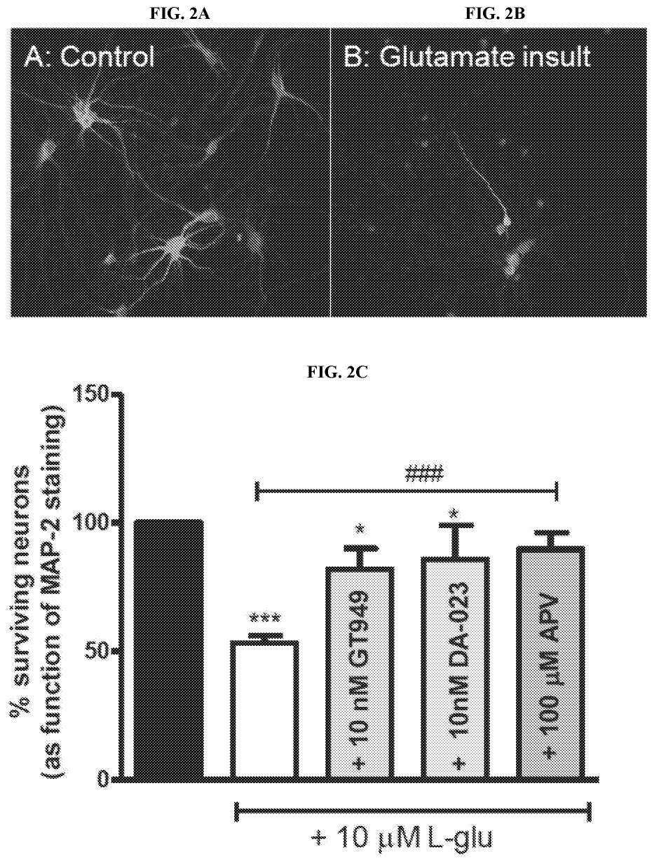

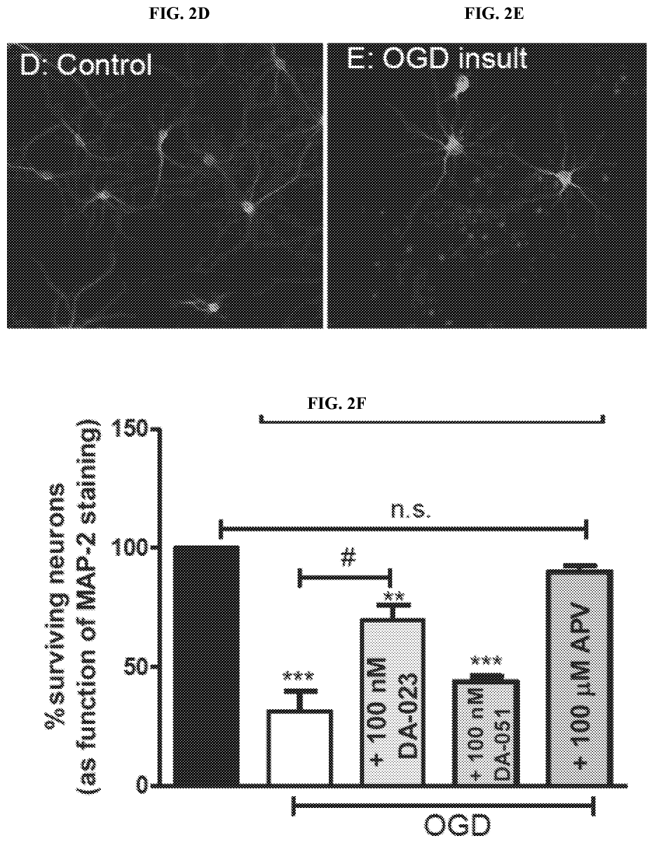

### DETAILED DESCRIPTION OF THE DISCLOSURE

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C≡CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “monovalent” as used herein refers to a substituent connecting via a single bond to a substituted molecule. When a substituent is monovalent, such as, for example, F or Cl, it is bonded to the atom it is substituting by a single bond.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “solvent” as used herein refers to a liquid that can dissolve a solid, liquid, or gas. Non-limiting examples of solvents are silicones, organic compounds, water, alcohols, ionic liquids, and supercritical fluids.

The term “independently selected from” as used herein refers to referenced groups being the same, different, or a mixture thereof, unless the context clearly indicates otherwise.

Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “room temperature” as used herein refers to a temperature of about 15° C. to 28° C.

The term “standard temperature and pressure” as used herein refers to 20° C. and 101 kPa.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, Pa.), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

As used herein, the term “DA-006” refers to 1-((1-benzyl-1H-tetrazol-5-yl)(4-methoxyphenyl)methyl)-4-cyclohexylpiperazine, or a tautomer, salt, or solvate thereof.

As used herein, the term “DA-015” refers to 3-((4-cyclohexylpiperazin-1-yl)(1-phenethyl-1H-tetrazol-5-yl)methyl)-6-methoxyquinolin-2(1H)-one, or a tautomer, salt, or solvate thereof.

As used herein, the term “DA-019” refers to 1-cyclohexyl-4-((1-phenethyl-1H-tetrazol-5-yl)(pyridin-3-yl)methyl)piperazine, or a tautomer, salt, or solvate thereof.

As used herein, the term “DA-023” refers to 1-methyl-4-((1-phenethyl-1H-tetrazol-5-yl)(pyridin-3-yl)methyl)piperazine, or a tautomer, salt, or solvate thereof.

As used herein, the term “DA-025” refers to 6-methoxy-3-((4-methylpiperazin-1-yl)(1-phenethyl-1H-tetrazol-5-yl)methyl)quinolin-2(1H)-one, or a tautomer, salt, or solvate thereof.

As used herein, the term “DA-035” refers to 4-((4-methylpiperazin-1-yl)(1-phenethyl-1H-tetrazol-5-yl)methyl)benzonitrile, or a tautomer, salt, or solvate thereof.

As used herein, the term “DA-036” refers to 1-((1-benzyl-1H-tetrazol-5-yl)(pyridin-3-yl)methyl)-4-cyclohexylpiperazine, or a tautomer, salt, or solvate thereof.

As used herein, the term “DA-037” refers to 1-((1-benzyl-1H-tetrazol-5-yl)(pyridin-3-yl)methyl)-4-methylpiperazine, or a tautomer, salt, or solvate thereof.

As used herein, the term “DA-038” refers to 4-((1-benzyl-1H-tetrazol-5-yl)(4-methylpiperazin-1-yl)methyl)benzonitrile, or a tautomer, salt, or solvate thereof.

As used herein, the term “DA-039” refers to 4-((4-cyclohexylpiperazin-1-yl)(1-phenethyl-1H-tetrazol-5-yl) methyl)benzonitrile, or a tautomer, salt, or solvate thereof.

As used herein, the term “DA-043” refers to 1-((4-methoxyphenyl)(1-phenethyl-1H-tetrazol-5-yl)methyl)-4-methylpiperazine, or a tautomer, salt, or solvate thereof.

As used herein, the term “DA-044” refers to 1-((4-fluorophenyl)(1-phenethyl-1H-tetrazol-5-yl)methyl)-4-methylpiperazine, or a tautomer, salt, or solvate thereof.

As used herein, the term “DA-045” refers to 4-((4-methylpiperazin-1-yl)(1-phenethyl-1H-tetrazol-5-yl)methyl)phenol, or a tautomer, salt, or solvate thereof.

As used herein, the term “DA-046” refers to 1-cyclohexyl-4-((4-fluorophenyl)(1-phenethyl-1H-tetrazol-5-yl)methyl)piperazine, or a tautomer, salt, or solvate thereof.

As used herein, the term “DA-050” refers to 1-cyclohexyl-4-((3,4-dimethoxyphenyl)(1-phenethyl-1H-tetrazol-5-yl)methyl)piperazine, or a tautomer, salt, or solvate thereof.

As used herein, the term “DA-058” refers to 1-methyl-4-((1-phenethyl-1H-tetrazol-5-yl)(pyridin-4-yl)methyl)piperazine, or a tautomer, salt, or solvate thereof.

As used herein, the term “NA-005” refers to N-(4-fluorophenethyl)-2-(4-methylpiperazin-1-yl)-2-(pyridin-3-yl)acetamide, or a tautomer, salt, or solvate thereof.

As used herein, the term “NA-014” refers to N-(4-chlorophenethyl)-2-(4-cyanophenyl)-2-(4-methylpiperazin-1-yl)acetamide, or a tautomer, salt, or solvate thereof.

As used herein, the term “VY-3-215” refers to 1-((1-benzyl-1H-tetrazol-5-yl)(6-fluoropyridin-3-yl)methyl)-4-methylpiperazine, or a tautomer, salt, or solvate thereof.

As used herein, the term “GT951” refers to 6-methoxy-3-((1-phenethyl-1H-tetrazol-5-yl)(4-(3-(trifluoromethyl)phenyl)piperazin-1-yl)methyl)quinolin-2(1H)-one, or a tautomer, salt, or solvate thereof.

As used herein, the term “VY-3-285” refers to 1-((6-fluoropyridin-3-yl)(1-phenethyl-1H-tetrazol-5-yl)methyl)-4-methylpiperazine, or a tautomer, salt, or solvate thereof.

As used herein, the term “VY-3-286” refers to 5-((4-methylpiperazin-1-yl)(1-phenethyl-1H-tetrazol-5-yl)methyl)pyridin-2-ol, or a tautomer, salt, or solvate thereof.

As used herein, the term “DA-055” refers to 1-((1-phenethyl-1H-tetrazol-5-yl)(pyridin-3-yl)methyl)piperazine, or a tautomer, salt, or solvate thereof.

As used herein, the term “VY-3-171” refers to N-(4-chlorobenzyl)-2-(4-methylpiperazin-1-yl)-2-(pyridin-3-yl)acetamide, or a tautomer, salt, or solvate thereof.

As used herein, the term “VY-3-209” refers to 4-((5-(4-fluorobenzylamino)-1,3,4-oxadiazol-2-yl)(4-methylpiperazin-1-yl)methyl)benzonitrile, or a tautomer, salt, or solvate thereof.

As used herein, the term “VY-3-136-1” refers to 1-((1-benzyl-1H-tetrazol-5-yl)(pyridin-3-yl)methyl)piperazine, or a tautomer, salt, or solvate thereof.

As used herein, the term “VY-3-136-2” refers to 1,4-bis((1-benzyl-1H-tetrazol-5-yl)(pyridin-3-yl)methyl)piperazine, or a tautomer, salt, or solvate thereof.

As used herein, the term “NA-010” refers to N-benzyl-2-(4-methylpiperazin-1-yl)-2-(pyridin-3-yl)acetamide, or a tautomer, salt, or solvate thereof.

As used herein, the term “NA-016” refers to 2-(4-cyanophenyl)-N-(4-fluorobenzyl)-2-(4-methylpiperazin-1-yl)acetamide, or a tautomer, salt, or solvate thereof.

As used herein, the term “NA-017” refers to 2-(4-cyanophenyl)-N-(4-methoxybenzyl)-2-(4-methylpiperazin-1-yl)acetamide, or a tautomer, salt, or solvate thereof.

As used herein, the term “NA-018” refers to N-benzyl-2-(4-cyanophenyl)-2-(4-methylpiperazin-1-yl)acetamide, or a tautomer, salt, or solvate thereof.

As used herein, the term “NA-019” refers to 2-(4-methylpiperazin-1-yl)-N-phenethyl-2-(pyridin-3-yl)acetamide, or a tautomer, salt, or solvate thereof.

Non-limiting abbreviations used herein include: AP-V, (DL)-2-amino-5-phosphonovaleric acid; D-PBS, Dulbecco's phosphate-buffered saline; EAAC1, excitatory amino acid carrier 1; EAATs, excitatory amino acid transporters; EAAT1-3, human excitatory amino acid transporter subtypes 1-3; EAAT2, human glutamate transporter 2; GLAST, glutamate and aspartate transporter; GLT-1, rat glutamate transporter 1; HP, hairpin loop; HSB, hybrid structure based; NaOH, sodium hydroxide; PBS-CM, D-PBS with 0.1 mM CaCl₂ and 1 mM MgCl₂ added; POPC, 1-palmitoyl-2-oleoyl-sn-glycero-3-phosphocholine; SDS, sodium dodecyl sulfate; TFB-TBOA, 3S)-3-[[3-[[4-(Trifluoromethyl) benzoyl]amino]phenyl]methoxy]-L-aspartic acid; TM, transmembrane; UCPH-101, 2-Amino-5,6,7,8-tetrahydro-4-(4-methoxyphenyl)-7-(naphthalen-1-yl)-5-oxo-4H-chromene-3-carbonitrile; WAY 213613, N-[4-(2-Bromo-4,5-difluorophenoxy)phenyl]-L-asparagine; WT, wild type; NBS, N-Bromosuccinimide; NIS, N-Iodosuccinimide; HATU, 1-[Bis(dimethylamino)methylene]-1H-1,2,3-triazolo[4,5-b]pyridinium 3-oxid hexafluorophosphate; DIPEA, N, N-Diisopropylethylamine; DCM, dicholoromethane; THF, tetrahydrofuran; DCC, N,N′-Dicyclohexylcarbodiimide; DMAP, 4-Dimethylaminopyridine.

Ranges: throughout this disclosure, various aspects can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

The compounds described herein can be prepared by the general schemes described herein, and/or using synthetic methods known by those skilled in the art. The following examples illustrate non-limiting embodiments of the compound(s) described herein and their preparation.

In one embodiment, the compound is of Formula I or Formula II, or an enantiomer, diastereoisomer, tautomer, salt, or solvate thereof:

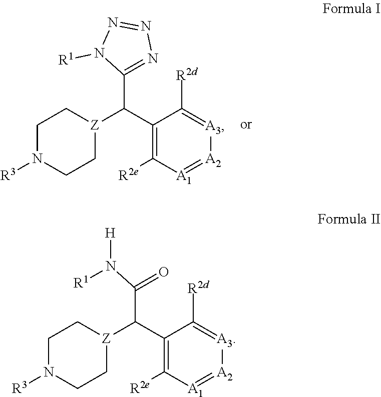

In the compound of Formula I or Formula II,
R¹ is selected from the group consisting of —C₁-C₆ alkyl, —C₁-C₆ heteroalkyl, —(C₀-C₃ alkyl)-(C₃-C₆ cycloalkyl), —(C₀-C₃ alkyl)-(C₄-C₁₀ heterocyclyl), —(C₀-C₃ alkyl)-(C₆-C₁₀ aryl) and —(C₀-C₃ alkyl)-(C₅-C₁₀ heteroaryl), wherein the alkyl, heteroalkyl, cycloalkyl, heterocyclyl, aryl and heteroaryl groups are independently optionally substituted;
A₁ is N or CR^{2a};
A₂ is N or CR^{2b};
A₃ is N or CR^{2c};
each occurrence of R^{2a}, R^{2b}, R^{2c}, R^{2d}, and R^{2e} is independently selected from the group consisting of H, tritium (³H), —C₁-C₆ alkyl, —OH, —C₁-C₆ alkoxy, halogen, —NH₂, —N(CH₃)₂, —C(═O)OH, trifluoromethyl, ≡C—N, —C(═O)O(C₁-C₄)alkyl, —C(═O)NH₂, —SO₂NH₂, —C(═NH)NH₂, and —NO₂;
R³ is selected from the group consisting of —(C═O)₀₋₁(C₁-C₆ alkyl), —(C═O)₀₋₁(C₁-C₆ heteroalkyl), —(C═O)₀₋₁(C₀-C₃ alkyl)-(C₃-C₆ cycloalkyl), —(C═O)₀₋₁(C₀-C₃ alkyl)-(C₄-C₁₀ heterocyclyl), —(C═O)₀₋₁(C₀-C₃ alkyl)-(C₆-C₁₀ aryl), —(C═O)₀₋₁(C₀-C₃ alkyl)-(C₅-C₁₀ heteroaryl), —(SO₂)₀₋₁(C₁-C₆ alkyl), —(SO₂)₀₋₁(C₁-C₆ heteroalkyl), —(SO₂)₀₋₁(C₀-C₃ alkyl)-(C₃-C₆ cycloalkyl), —(SO₂)₀₋₁(C₀-C₃ alkyl)-(C₄-C₁₀ heterocyclyl), —(SO₂)₀₋₁(C₀-C₃ alkyl)-(C₆-C₁₀ aryl) and —(SO₂)₀₋₁(C₀-C₃ alkyl)-(C₅-C₁₀ heteroaryl), wherein the alkyl, heteroalkyl, cycloalkyl, heterocyclyl, aryl and heteroaryl groups are independently optionally substituted; and
Z is N or CH.

In various embodiments, one or more hydrogen atoms in R¹ are replaced with deuterium (2H) and/or tritium (³H).

In various embodiments, the compound is of Formula I-1 or I-2:

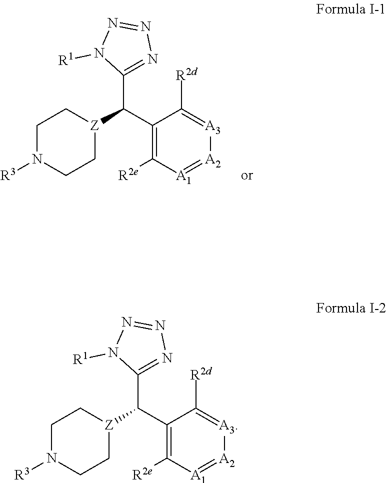

In various embodiments, the compound is of Formula II-1 or II-2:

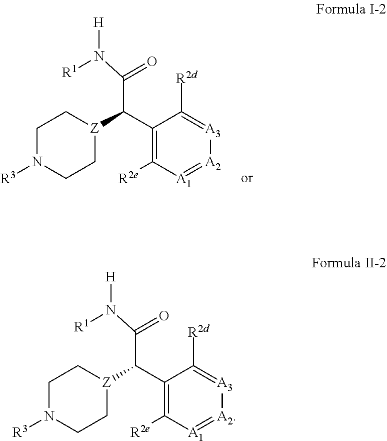

In various embodiments, Z is N. In various embodiments, only one of A₁, A₂, and A₃ is N.

In various embodiments, R¹ is selected from —(C₀-C₃ alkyl)-(C₃-C₆ cycloalkyl), —(C₀-C₃ alkyl)-(C₄-C₁₀ heterocyclyl), —(C₀-C₃ alkyl)-(C₆-C₁₀ aryl) and —(C₀-C₃ alkyl)-(C₅-C₁₀ heteroaryl), wherein the alkyl, heteroalkyl, cycloalkyl, heterocyclyl, aryl and heteroaryl groups are independently optionally substituted.

In various embodiments, R¹ is selected from —(C₀-C₃ alkyl)-(C₃-C₆ cycloalkyl) and —(C₀-C₃ alkyl)-(C₆-C₁₀ aryl), wherein the alkyl, cycloalkyl, and aryl groups are independently optionally substituted. In various embodiments, R¹ is —(C₀-C₃ alkyl)-(C₆-C₁₀ aryl), wherein the alkyl and aryl groups are independently optionally substituted. In various embodiments, R¹ is —(C₀-C₃ alkyl)-phenyl, wherein the phenyl group is optionally substituted by 1 to 5 groups selected from the group consisting of tritium (³H), —C₁-C₆ alkyl, —OH, —C₁-C₆ alkoxy, halogen, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂.

In various embodiments, A₁ is N. In various embodiments, A₂ is N. In various embodiments, A₃ is N. In various embodiments, A₁ is CR^{2a}, A₂ is CR^{2b}, and A₃ is CR^{2e}.

In various embodiments, each occurrence of R^{2a}, R^{2b}, R^{2c}, R^{2d}, and R^{2e} is independently selected from the group consisting of H, deuterium (2H), tritium (³H), —OH, —C₁-C₆ alkoxy, halogen, trifluoromethyl, and —C≡N.

In various embodiments, R³ is selected from the group consisting of —(C═O)₀₋₁(C₁-C₆ alkyl), —(C═O)₀₋₁(C₁-C₆ heteroalkyl), and —(C═O)₀₋₁(C₀-C₃ alkyl)-(C₃-C₆ cycloalkyl), wherein the alkyl, heteroalkyl, and cycloalkyl are independently optionally substituted. In various embodiments, R³ is (C₁-C₆ alkyl) or (C₀-C₃ alkyl)-(C₃-C₆ cycloalkyl). In various embodiments, R³ is methyl or cyclohexyl.

In one embodiment, compounds of Formula I can be prepared according to Scheme 1.

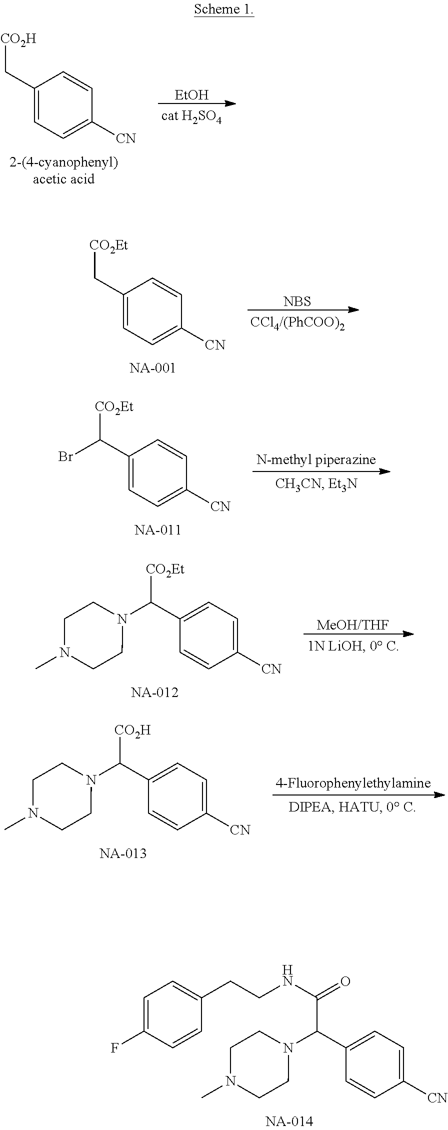

In various embodiments, compounds of Formula I can be prepared in an enantioselective manner according to Scheme 2.

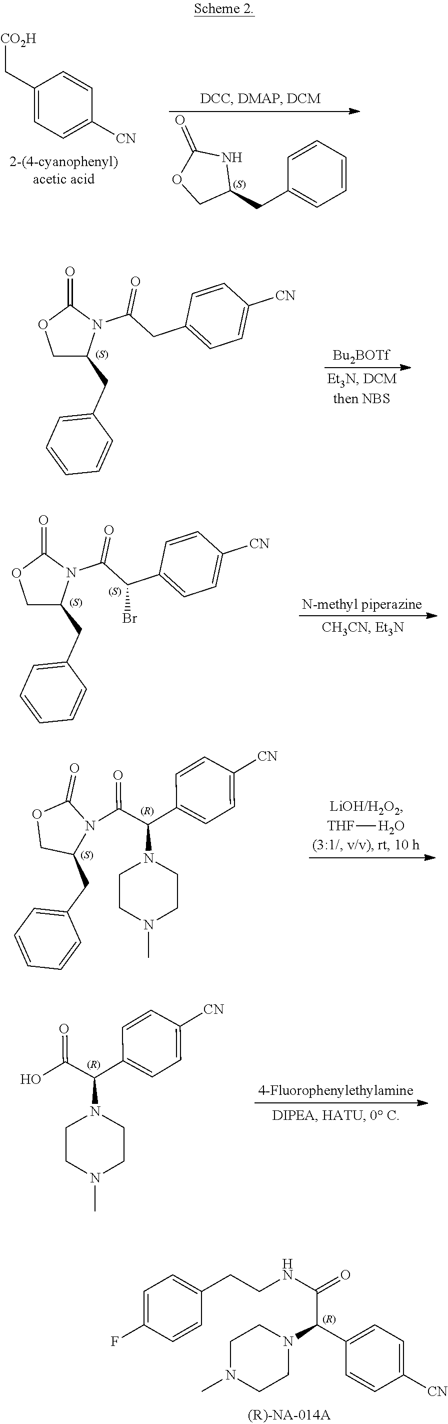

In various embodiments, an isotopically labeled compound of Formula I can be prepared according to Scheme 3.

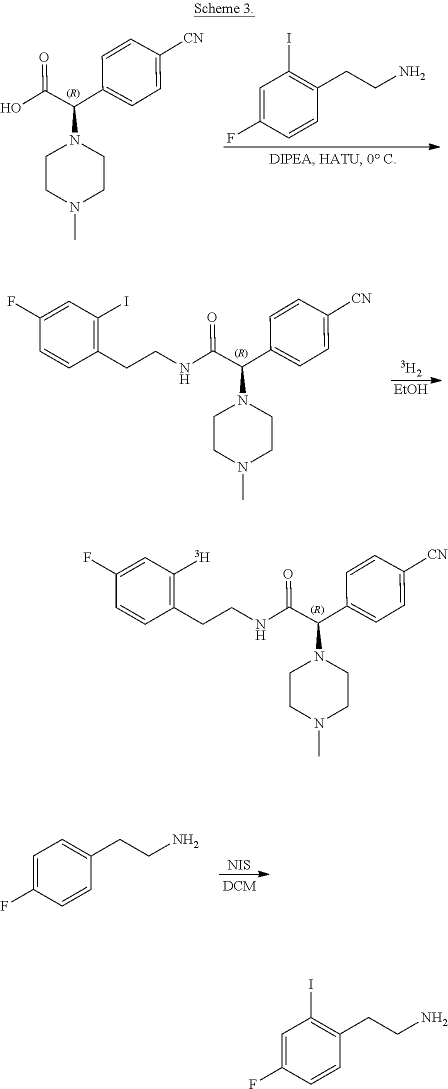

In various embodiments, the compounds of Formula I are EAAT2 activators with an EC₅₀ of at least, less than, or greater than about 0.01 nM to about 300 nM. In various embodiments, the compounds of Formula I are EAAT2 activators with an EC₅₀ of at least, less than, or greater than about 0.01 nM, 0.05 nM, 0.1 nM, 0.5 nM, 1 nM, 2 nM, 3 nM, 4 nM, 5 nM, 10 nM, 15 nM, 20 nM, 25 nM, 30 nM, 40 nM, 50 nM, 60 nM, 70 nM, 80 nM, 90 nM, 100 nM, 125 nM, 150 nM, 175 nM, 200 nM, 225 nM, 250 nM, 275 nM, or 300 nM.

In various embodiments, the compounds of Formula II are EAAT2 activators with an EC₅₀ of at least, less than, or greater than about 0.01 nM to about 300 nM. In various embodiments, the compounds of Formula II are EAAT2 activators with an EC₅₀ of at least, less than, or greater than about 0.01 nM, 0.05 nM, 0.1 nM, 0.5 nM, 1 nM, 2 nM, 3 nM, 4 nM, 5 nM, 10 nM, 15 nM, 20 nM, 25 nM, 30 nM, 40 nM, 50 nM, 60 nM, 70 nM, 80 nM, 90 nM, 100 nM, 125 nM, 150 nM, 175 nM, 200 nM, 225 nM, 250 nM, 275 nM, or 300 nM.

In various embodiments, the compound is selected from the group consisting of:

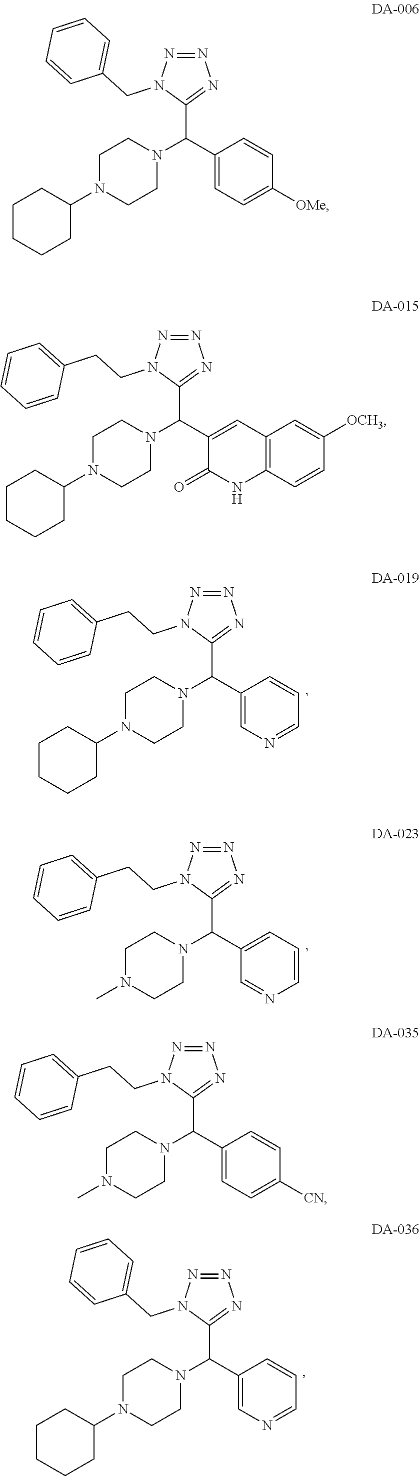

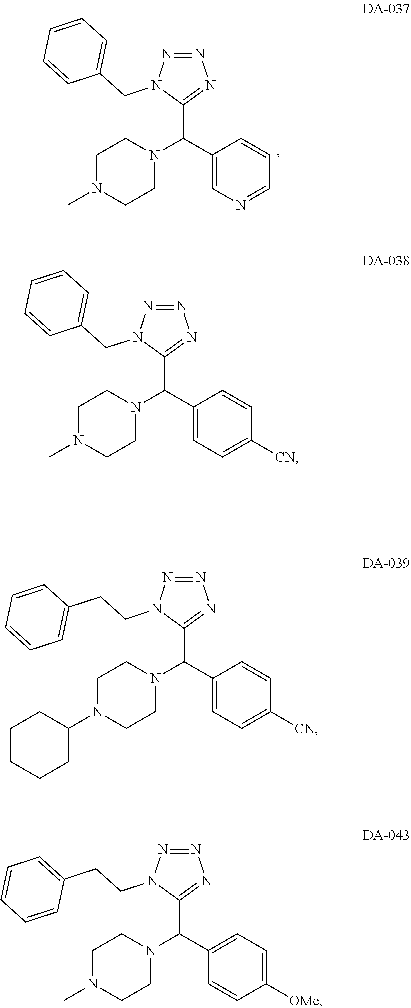 [SMILES: CN1CCN(C(c2cccnc2)c2nnnn2Cc2ccccc2)CC1 ; "," ; CN1CCN(C(c2ccc(C#N)cc2)c2nnnn2Cc2ccccc2)CC1 ; N#Cc1ccc(C(c2nnnn2CCc2ccccc2)N2CCN(C3CCCCC3)CC2)cc1 ; COc1ccc(C(c2nnnn2CCc2ccccc2)N2CCN(C)CC2)cc1]

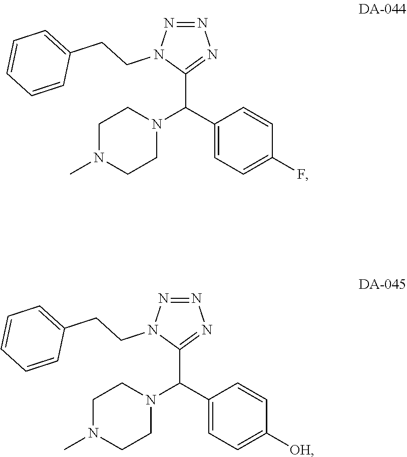 [SMILES: CN1CCN(C(c2ccc(F)cc2)c2nnnn2CCc2ccccc2)CC1 ; CN1CCN(C(c2ccc(O)cc2)c2nnnn2CCc2ccccc2)CC1]

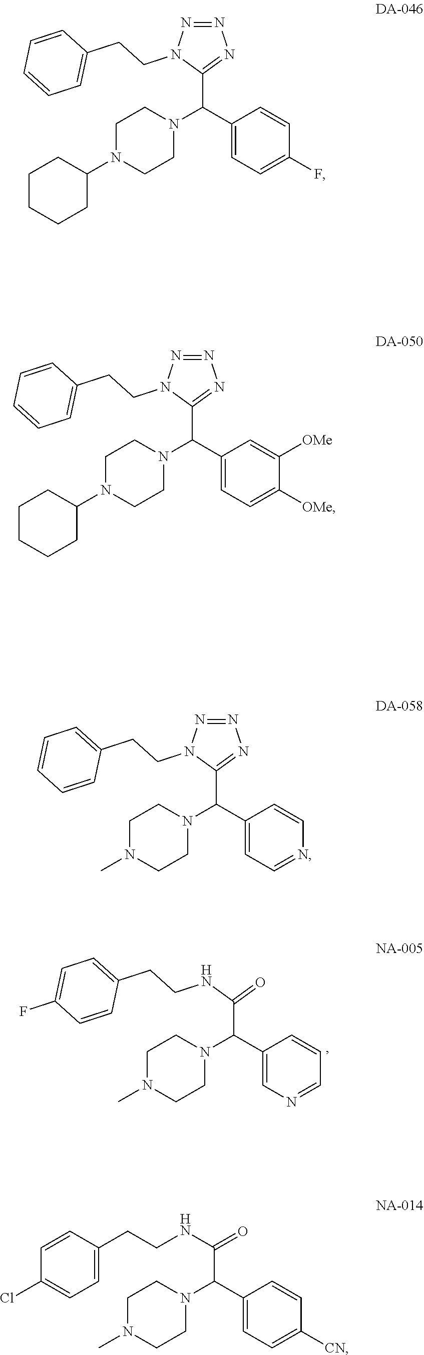 [SMILES: Fc1ccc(C(c2nnnn2CCc2ccccc2)N2CCN(C3CCCCC3)CC2)cc1 ; COc1ccc(C(c2nnnn2CCc2ccccc2)N2CCN(C3CCCCC3)CC2)cc1OC ; CN1CCN(C(c2ccncc2)c2nnnn2CCc2ccccc2)CC1 ; CN1CCN(C(C(=O)NCCc2ccc(F)cc2)c2cccnc2)CC1 ; "," ; CN1CCN(C(C(=O)NCCc2ccc(Cl)cc2)c2ccc(C#N)cc2)CC1]

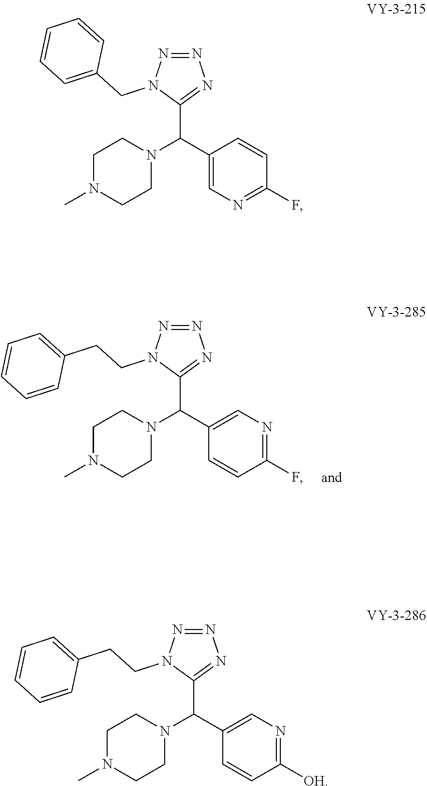 [SMILES: CN1CCN(C(c2ccc(F)nc2)c2nnnn2Cc2ccccc2)CC1 ; CN1CCN(C(c2ccc(F)nc2)c2nnnn2CCc2ccccc2)CC1 ; "and" ; CN1CCN(C(c2ccc(O)nc2)c2nnnn2CCc2ccccc2)CC1]

In various embodiments, the compounds of the disclosure include compounds in Table 1.

TABLE 1

| | | | Mass |
|---|---|---|---|
| No. | Structure | IUPAC Nomenclature | Characterization |
| 1 | 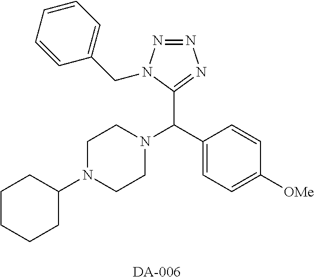 | 1-((1-benzyl-1H-tetrazol-5-yl)(4- methoxyphenyl)methyl)-4- cyclohexylpiperazine | MS (ESI): m/z 447.4 [M + 1]⁺; HRMS (ESI m/z) for C₂₆H₃₅ON₆, calcd 447.28669, found 447.28611 [M + 1]⁺. |
| 2 | 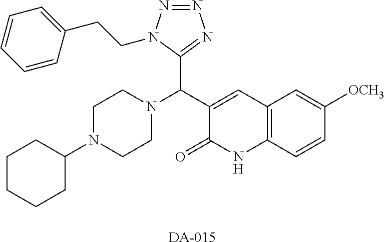 | 3-((4-cyclohexylpiperazin-1-yl)(1- phenethyl-1H-tetrazol-5-yl)methyl)-6- methoxyquinolin-2(1H)-one | MS (ESI): m/z 528.4 [M + 1]⁺; HRMS (ESI m/z) for C₃₀H₃₈O₂N₇, calcd 528.30815, found 528.30810 [M + 1]⁺ |
| 3 | 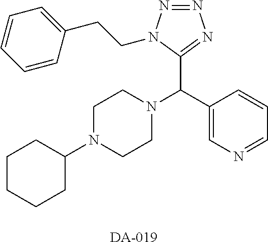 | 1-cyclohexyl-4-((1-phenethyl-1H-tetrazol- 5-yl)(pyridin-3-yl)methyl)piperazine | MS (ESI): m/z 432.3 [M + 1]⁺; HRMS (ESI m/z) for C₂₅H₃₄N₇, calcd 432.28702, found 432.28664 [M + 1]⁺. |
| 4 | 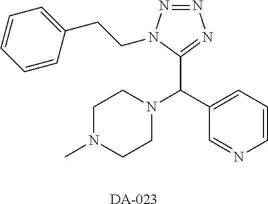 | 1-methyl-4-((1-phenethyl-1H-tetrazol-5- yl)(pyridin-3-yl)methyl)piperazine | MS (ESI): m/z 364.3 [M + 1]⁺; HRMS (ESI m/z) for C₂₀H₂₆N₇, calcd 364.22442, found 364.22399 [M + 1]⁺. |
| 5 | 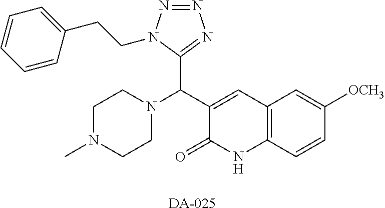 | 6-methoxy-3-((4-methylpiperazin-1- yl)(1-phenethyl-1H-tetrazol-5- yl)methyl)quinolin-2(1H)-one | MS (ESI): m/z 460.3 [M + 1]⁺; HRMS (ESI m/z) for C₂₅H₃₀O₂N₇, calcd 460.24555, found 460.24530 [M + 1]⁺. |
| 6 | 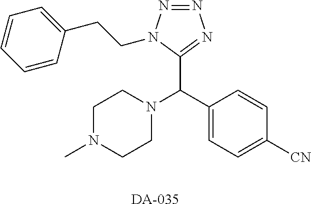 | 4-((4-methylpiperazin-1-yl)(1- phenethyl-1H-tetrazol-5- yl)methyl)benzonitrile | MS (ESI): m/z 388.4 [M + 1]⁺; HRMS (ESI m/z) for C₂₂H₂₆N₇, calcd 388.22442, found 388.22472 [M + 1]⁺. |
| 7 | 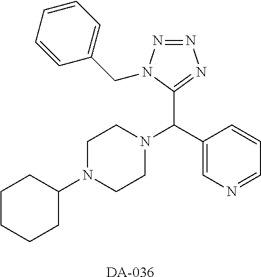 | 1-((1-benzyl-1H-tetrazol-5-yl)(pyridin- 3-yl)methyl)-4-cyclohexylpiperazine | MS (ESI): m/z 418.4 [M + 1]⁺; HRMS (ESI m/z) for C₂₄H₃₂N₇, calcd 418.47137, found 418.37157. |
| 8 | 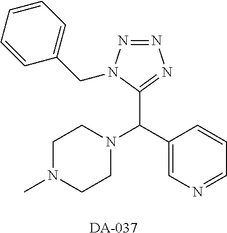 | 1-((1-benzyl-1H-tetrazol-5-yl)(pyridin- 3-yl)methyl)-4-methylpiperazine | MS (ESI): m/z 350.4 [M + 1]⁺; HRMS (ESI m/z) for C₁₉H₂₄N₇, calcd 350.20877, found 350.20849 [M + 1]⁺. |
| 9 | 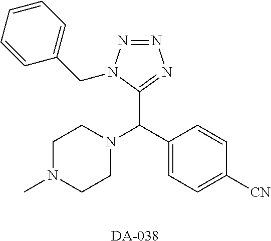 | 4-((1-benzyl-1H-tetrazol-5-yl)(4- methylpiperazin-1- yl)methyl)benzonitrile | MS (ESI): m/z 374.4 [M + 1]⁺; HRMS (ESI m/z) for C₂₁H₂₄N₇, calcd 374.20877, found 374.20824 [M + 1]⁺. |
| 10 | 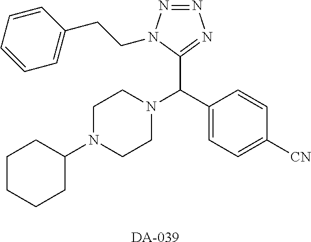 | 4-((4-cyclohexylpiperazin-1-yl)(1- phenethyl-1H-tetrazol-5-yl) methyl)benzonitrile | LCMS (ESI): m/z 456 [M + 1]⁺. |
| 11 | 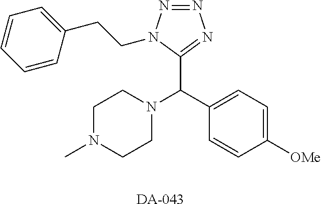 | 1-((4-methoxyphenyl)(1-phenethyl-1H- tetrazol-5-yl)methyl)-4- methylpiperazine | MS (ESI): m/z 393.4 [M + 1]⁺; HRMS (ESI m/z) for C₂₂H₂₉ON₆, calcd 393.23974, found 393.23914 [M + 1]⁺. |
| 12 | 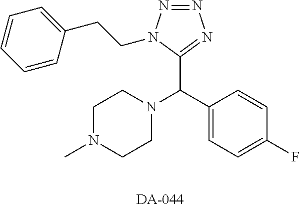 | 1-((4-fluorophenyl)(1-phenethyl-1H- tetrazol-5-yl)methyl)-4- methylpiperazine | MS (ESI): m/z 381.4 [M + 1]⁺; HRMS (ESI m/z) for C₂₁H₂₆N₇F, calcd 381.21975, found 381.21904 [M + 1]⁺. |
| 13 | 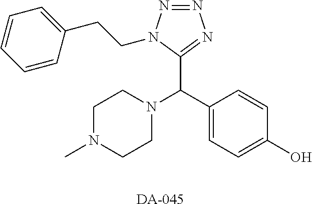 | 4-((4-methylpiperazin-1-yl)(1- phenethyl-1H-tetrazol-5- yl)methyl)phenol | MS (ESI): m/z 379.4 [M + 1]⁺; HRMS (ESI m/z) for C₂₁H₂₇ON₆, calcd 379.22409, found 379.22550 [M + 1]⁺. |
| 14 | 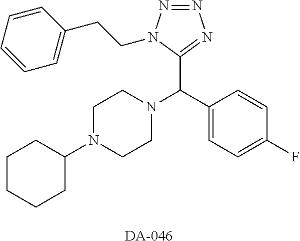 | 1-cyclohexyl-4-((4-fluorophenyl)(1- phenethyl-1H-tetrazol-5- yl)methyl)piperazine | MS (ESI): m/z 449.4 [M + 1]⁺; HRMS (ESI m/z) for C₂₆H₃₄N₆F, calcd 449.28235, found 449.28277 [M + 1]⁺. |
| 15 | 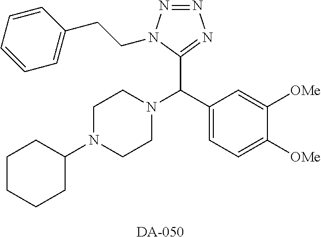 | 1-cyclohexyl-4-((3,4- dimethoxyphenyl)(1-phenethyl-1H- tetrazol-5-yl)methyl)piperazine | MS (ESI): m/z 491.4 [M + 1]⁺; HRMS (ESI m/z) for C₂₈H₃₉O₂N₆, calcd 491.31290, found 491.31345. [M + 1]⁺. |
| 16 | 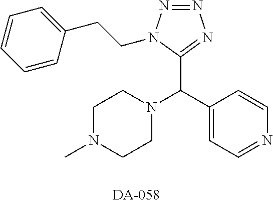 | 1-methyl-4-((1-phenethyl-1H-tetrazol-5- yl)(pyridin-4-yl)methyl)piperazine | MS (ESI): m/z 350.5 [M + 1]⁺; HRMS (ESI m/z) for C₁₉H₂₄N₇, calcd 350.20877, found 350.20892. [M + 1]⁺. |
| 17 | 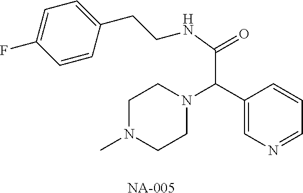 | N-(4-fluorophenethyl)-2-(4- methylpiperazin-1-yl)-2-(pyridin-3- yl)acetamide | LCMS (ESI): m/z 357 [M + 1]⁺. |
| 18 | 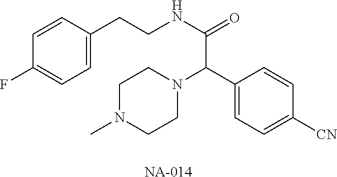 | N-(4-chlorophenethyl)-2-(4- cyanophenyl)-2-(4-methylpiperazin-1- yl)acetamide | LCMS (ESI): m/z 381 [M + 1]⁺. |
| 19 | 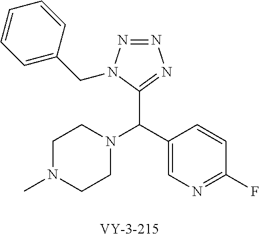 | 1-((1-benzyl-1H-tetrazol-5-yl)(6- fluoropyridin-3-yl)methyl)-4- methylpiperazine | LCMS (ESI): m/z 368 [M + 1]⁺. |
| 20 | 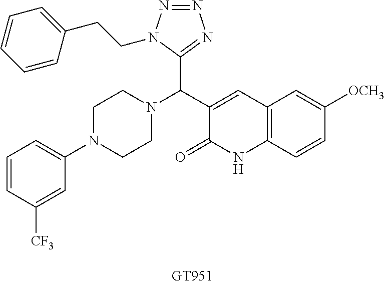 [SMILES: COc1ccc2[nH]c(=O)c(C(c3nnnn3CCc3ccccc3)N3CCN(c4cccc(C(F)(F)F)c4)CC3)cc2c1] | 6-methoxy-3-((1-phenethyl-1H-tetrazol- 5-yl)(4-(3- (trifluoromethyl)phenyl)piperazin-1- yl)methyl)quinolin-2(1H)-one | LCMS (ESI): m/z 590 [M + 1]⁺. |
| 21 | 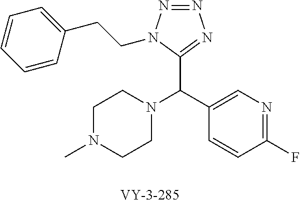 | 1-((6-fluoropyridin-3-yl)(1-phenethyl- 1H-tetrazol-5-yl)methyl)-4- methylpiperazine | LCMS (ESI): m/z 382 [M + 1]⁺. |
| 22 | 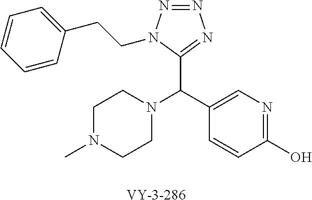 | 5-((4-methylpiperazin-1-yl)(1- phenethyl-1H-tetrazol-5- yl)methyl)pyridin-2-ol | LCMS (ESI): m/z 380 [M + 1]⁺. |

In various embodiments, the compounds of the disclosure include compounds in Table 2.

TABLE 2

| | | | |
|---|---|---|---|
| 29 | 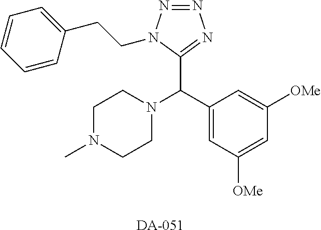 | 1-((3,5-dimethoxyphenyl)(1-phenethyl-1H- tetrazol-5-yl)methyl)-4-methylpiperazine | LCMS (ESI): m/z 423 [M + 1]⁺ |
| 30 | 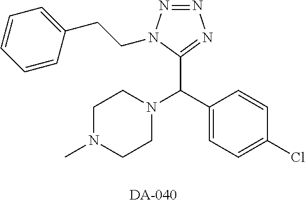 | 1-((4-chlorophenyl)(1-phenethyl-1H- tetrazol-5-yl)methyl)-4-methylpiperazine | MS (ESI): m/z 397.4 [M + 1]⁺; HRMS (ESI m/z) for C₂₁H₂₆N₆Cl, calcd 397.19020, found 397.18958. [M + 1]⁺. |
| 31 | 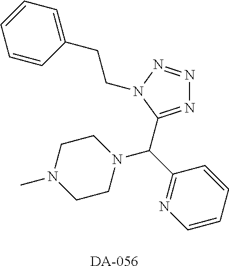 | 1-methyl-4-((1-phenethyl-1H-tetrazol-5- yl)(pyridin-2-yl)methyl)piperazine | MS (ESI): m/z 364.4 [M + 1]⁺; HRMS (ESI m/z) for C₂₀H₂₆N₇, calcd 364.22442, found 364.22497 [M + 1]⁺. |
| 32 | 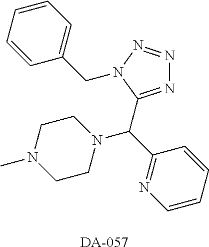 | 1-((1- benzyl-1H-tetrazol-5-yl)(pyridin- 2-yl)methyl)-4-methylpiperazine | MS (ESI): m/z 350.5 [M + 1]⁺; HRMS (ESI m/z) for C₁₉H₂₄N₇, calcd 350.20877, found 350.20953. [M + 1]⁺. |
| 33 | 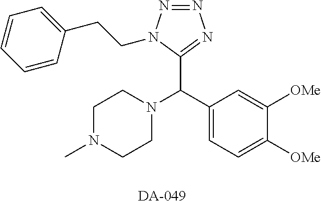 | 1-((3,4-dimethoxyphenyl)(1-phenethyl- 1H-tetrazol-5-yl)methyl)-4- methylpiperazine | MS (ESI): m/z 423.5 [M + 1]⁺; HRMS (ESI m/z) for C₂₃H₃₁O₂N₆, calcd 423.25030, found 423.25025 [M + 1]⁺. |
| 34 | 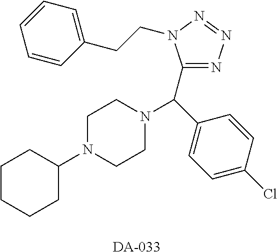 | 1-((4-chlorophenyl)(1-phenethyl-1H- tetrazol-5-yl)methyl)-4- cyclohexylpiperazine | MS (ESI): m/z 465.4 [M + 1]⁺; HRMS (ESI m/z) for C₂₆H₃₄N₆Cl, calcd 465.25280, found 465.25381. [M + 1]⁺. |
| 35 | 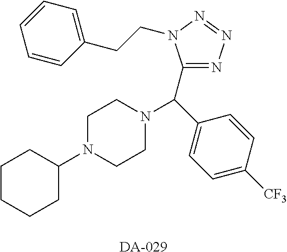 | 1-cyclohexyl-4-((1-phenethyl-1H- tetrazol-5-yl)(4- (trifluoromethyl)phenyl)methyl)piperazine | LCMS (ESI): m/z 499 [M + 1]⁺ |
| 36 | 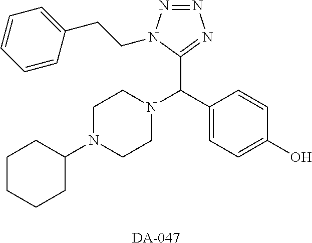 | 4-((4-cyclohexylpiperazin-1-yl)(1- phenethyl-1H-tetrazol-5- yl)methyl)phenol | MS (ESI): m/z 447.5 [M + 1]⁺; HRMS (ESI m/z) for C₂₆H₃₅ON₆, calcd 447.28669, found 447.28609. [M + 1]⁺. |
| 37 | 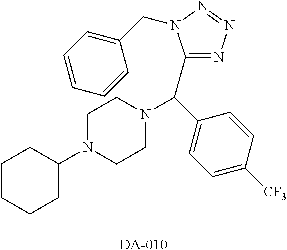 | 1-((1-benzyl-1H-tetrazol-5-yl)(4- (trifluoromethyl)phenyl)methyl)-4- cyclohexylpiperazine | MS (ESI): m/z 485.4 [M + 1]⁺; HRMS (ESI m/z) for C₂₆H₃₂N₆F₃, calcd 485.26351, found 485.26307. [M + 1]⁺. |
| 38 | 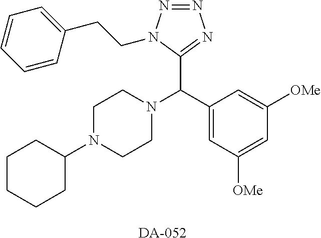 | 1-cyclohexyl-4-((3,5- dimethoxyphenyl)(1-phenethyl-1H- tetrazol-5-yl)methyl)piperazine | LCMS (ESI): m/z 491 [M + 1]⁺. |
| 39 | 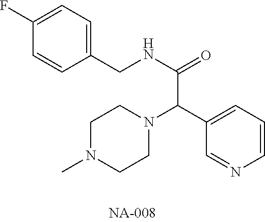 [SMILES: CN1CCN(C(C(=O)NCc2ccc(F)cc2)c2cccnc2)CC1] | N-(4-fluorobenzyl)-2-(-4 methylpiperazin-1-yl)-2-(pyridin-3- yl)acetamide | LCMS (ESI): m/z 343 [M + 1]⁺. |
| 40 | 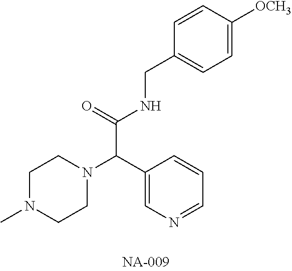 [SMILES: COc1ccc(CNC(=O)C(c2cccnc2)N2CCN(C)CC2)cc1] | N-(4-methoxybenzyl)-2-(4-methylpiperazin- 1-yl)-2-(pyridin-3-yl)acetamide | LCMS (ESI): m/z 355 [M + 1]⁺. |
| 41 | 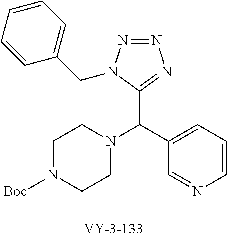 | tert-butyl 4-((1-benzyl-1H-tetrazol-5- yl)(pyridin-3-yl)methyl)piperazine-1- carboxylate | LCMS (ESI): m/z 436 [M + 1]⁺. |
| 42 | 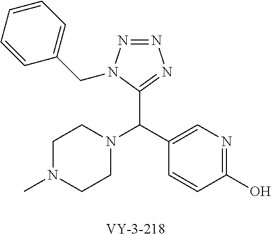 [SMILES: CN1CCN(C(c2ccc(O)nc2)c2nnnn2Cc2ccccc2)CC1 ; "VY-3-218"] | 5-((1-benzyl-1H-tetrazol-5-yl)(4- methylpiperazin-1-yl)methyl)pyridin-2- ol | LCMS (ESI): m/z 366 [M + 1]⁺. |
| 43 | 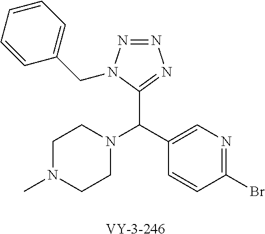 [SMILES: CN1CCN(C(c2ccc(Br)nc2)c2nnnn2Cc2ccccc2)CC1 ; "VY-3-246"] | 1-((1-benzyl-1H-tetrazol-5-yl)(6- bromopyridin-3-yl)methyl)-4- methylpiperazine | LCMS (ESI): m/z 430 [M + 2]⁺. |
| 44 | 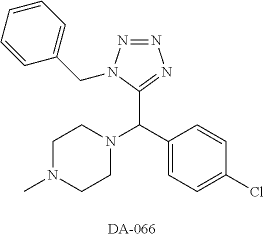 | 1-((4-chlorophenyl)(1-benzyl-1H- tetrazol-5-yl)methyl)-4- methylpiperazine | LCMS (ESI): m/z 383 [M + 1]⁺. |
| 45 | 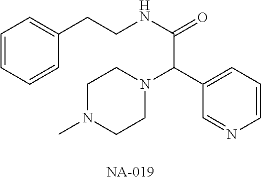 | 2-(4-methylpiperazin-1-yl)-N- phenethyl-2-(pyridin-3-yl)acetamide | LCMS (ESI): m/z 339 [M + 1]⁺. |
| 46 | 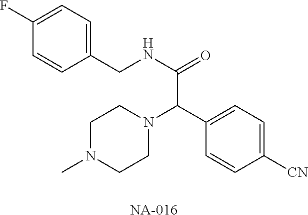 | 2-(4-cyanophenyl)-N-(4-fluorobenzyl)- 2-(4-methylpiperazin-1-yl)acetamide | LCMS (ESI): m/z 467 [M + 1]⁺. |
| 47 | 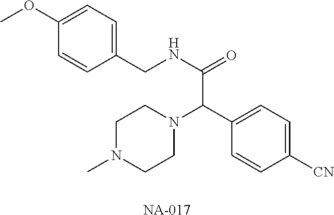 | 2-(4-cyanophenyl)-N-(4- methoxybenzyl)-2-(4-methylpiperazin- 1-yl)acetamide | LCMS (ESI): m/z 379 [M + 1]⁺. |
| 48 | 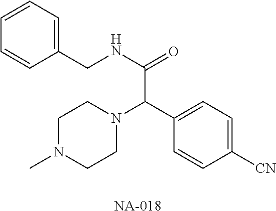 | N-benzyl-2-(4-cyanophenyl)-2-(4- methylpiperazin-1-yl)acetamide | LCMS (ESI): m/z 349 [M + 1]⁺. |

In various embodiments, the compounds of the disclosure include compounds in Table 3.

TABLE 3

| | | | |
|---|---|---|---|
| 23 | 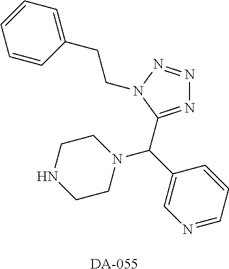 | 1-((1-phenethyl-1H-tetrazol-4- yl)(pyridin-3-yl)methyl)piperazine | LCMS (ESI): m/z 350 [M + 1]⁺ |
| 24 | 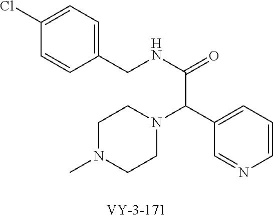 | N-(4-chlorobenzyl)-2-(4- methylpiperazin-1-yl)-2-(pyridin-3- yl)acetamide | LCMS (ESI): m/z 373 [M + 1]⁺. |
| 26 | 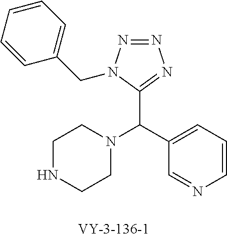 | 1-((1-benzyl-1H-tetrazol-5-yl)(pyridin- 3-yl)methyl)piperazine | LCMS (ESI): m/z 336 [M + 1]⁺. |
| 28 | 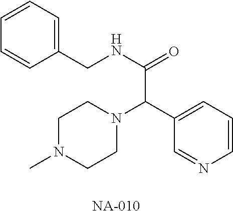 | N-benzyl-2-(4-methylpiperazin-1-yl)-2- (pyridin-3-yl)acetamide | LCMS (ESI): m/z 325 [M + 1]⁺. |

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S.

In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

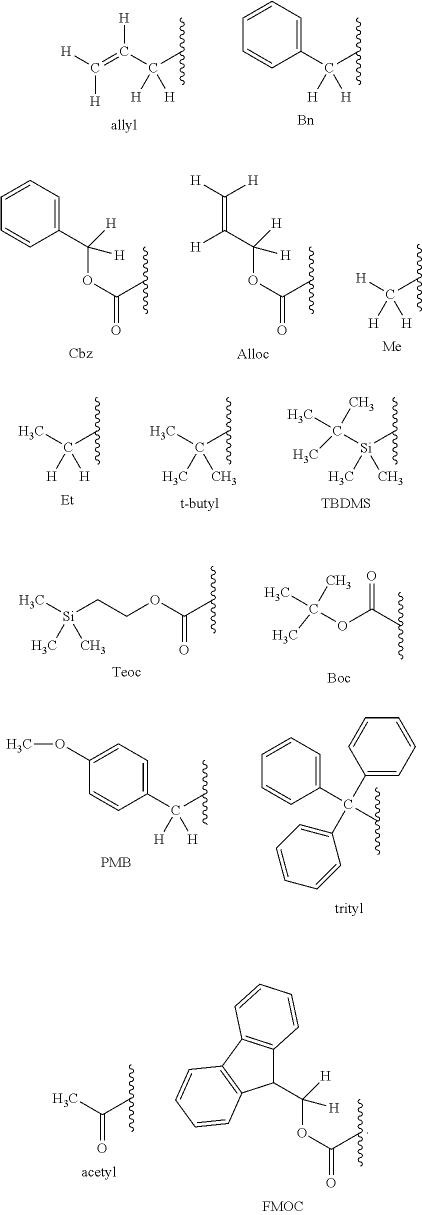

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### Compositions

The compositions containing the compound(s) described herein include a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration.

### Methods of Treatment

Compounds of Formula I are useful in methods of treating or ameliorating a disease or disorder that is caused, induced or characterized by abnormal reduction in glutamate transporter activity or abnormal increase in extracellular CNS glutamate concentration in a subject. The method includes administering to the subject a therapeutically effective amount of at least one compound described herein, or an enantiomer, diastereoisomer, tautomer, salt, or solvate thereof. In certain embodiments, the disease or disorder is selected from the group consisting of ischemia, seizure, traumatic brain injury, stroke, epilepsy, schizophrenia, and neurodegenerative diseases or disorders (such as, but not limited to, Parkinson's disease, Alzheimer's disease, Huntington's disease, amyotrophic lateral sclerosis (ALS), and ALS-parkinsonism dementia complex, and HIV-associated neurocognitive disorder (HAND), neuropathic pain, mental health disorders and drug abuse/relapse.

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional therapeutic agent that treats or prevents a disease or disorder selected from the group consisting of ischemia, seizure, traumatic brain injury, stroke, epilepsy, schizophrenia, and neurodegenerative diseases or disorders (such as, but not limited to, Parkinson's disease, Alzheimer's disease, Huntington's disease, amyotrophic lateral sclerosis (ALS), and ALS-parkinsonism dementia complex, HIV-associated neurocognitive disorder (HAND) neuropathic pain, mental health disorders and drug abuse/relapse.

In certain embodiments, administering the compound(s) described herein to the subject allows for administering a lower dose of the additional therapeutic agent as compared to the dose of the additional therapeutic agent alone that is required to achieve similar results in treating or preventing a disease or disorder described as being treatable with at least one compound described herein in the subject. For example, in certain embodiments, the compound(s) described herein enhance(s) the activity of the additional therapeutic compound, thereby allowing for a lower dose of the additional therapeutic compound to provide the same effect.

In certain embodiments, the compound(s) described herein and the therapeutic agent are co-administered to the subject. In other embodiments, the compound(s) described herein and the therapeutic agent are coformulated and co-administered to the subject.

In certain embodiments, the subject is a mammal. In other embodiments, the mammal is a human.

In various embodiments, the compounds of Formula I have the EC₅₀ and efficacy against EAAT1, EAAT2, and EAAT3 listed in Table 4. In Table 4, the top number for each compound is the EC₅₀ and the bottom number, which is expressed as a percentage, is the efficacy against the listed EAAT receptor.

TABLE 4

| | EAAT1 EC₅₀ (nM) | EAAT2 EC₅₀ (nM) | EAAT3EC₅₀ (nM) | |
|---|---|---|---|---|
| Compound | Efficacy | Efficacy | Efficacy | Structure |
| NA-005 | 57.95 ± 11.20 189 ± 9.89% | 10.30 ± 11.20 151.50 ± 51.20% | 4.33 ± 7.50 233.50 ± 55.86% | 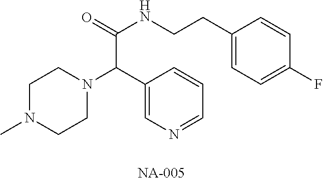 |
| VY-3-285 | 2.75 ± 3.18 222 ± 55.72% | 0.73 ± 0.94 156 ± 4.24% | 2.54 ± 3.47 188.2 ± 5.09% | 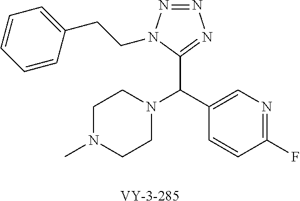 |
| VY-3-286 | 2.00 ± 1.41 160.25 ± 27.22% | 0.95 ± 0.07 186.50 ± 24.70% | 1.55 ± 2.05 130.50 ± 2.12% | 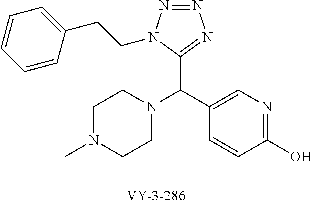 |
| DA-050 | 46.00 ± 36.76 150.00 ± 21.21% | 0.50 ± 0.56 261.50 ± 40.30% | No Effect | 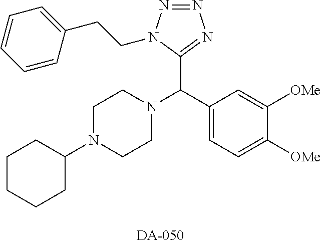 |
| DA-058 | 2.24 ± 1.07 186.0 ± 41.01% | 0.40 ± 0.42 200.50 ± 58.60% | No Effect | 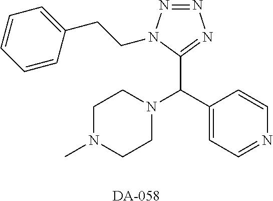 |
| NA-014 | No Effect | 3.50 ± 4.40 167.30 ± 8.30% | No Effect | 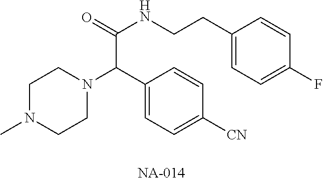 |

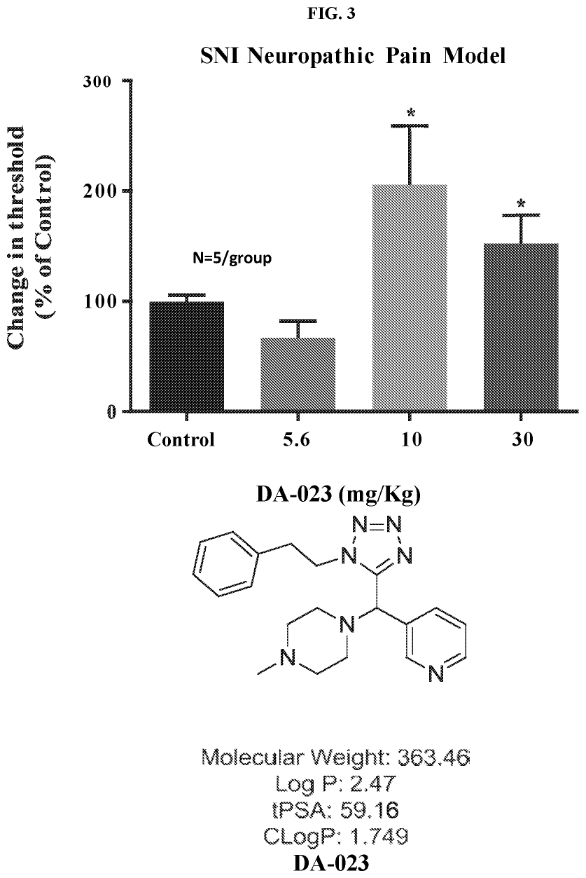

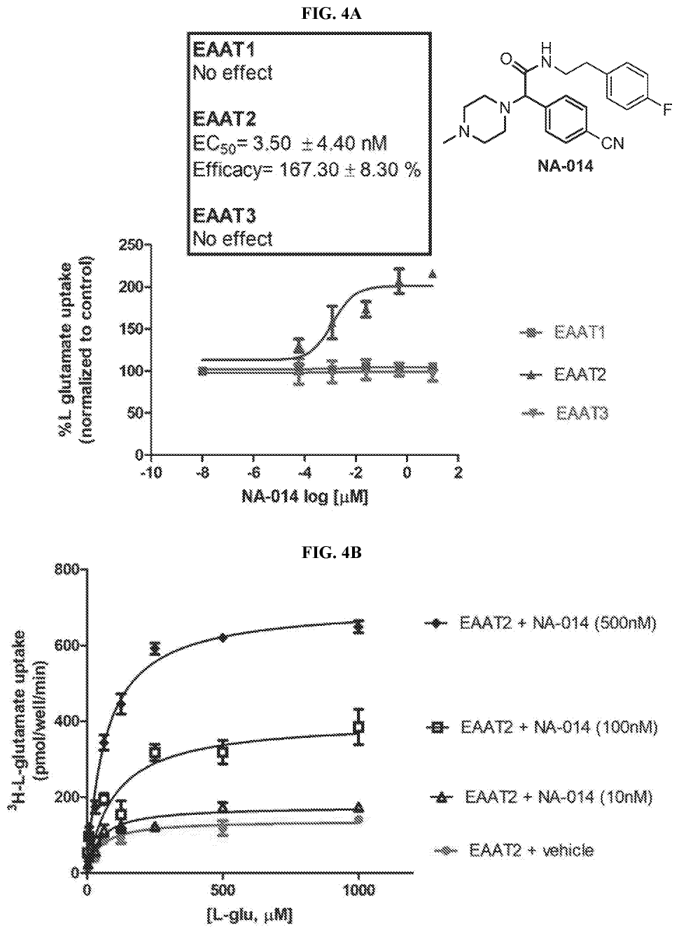

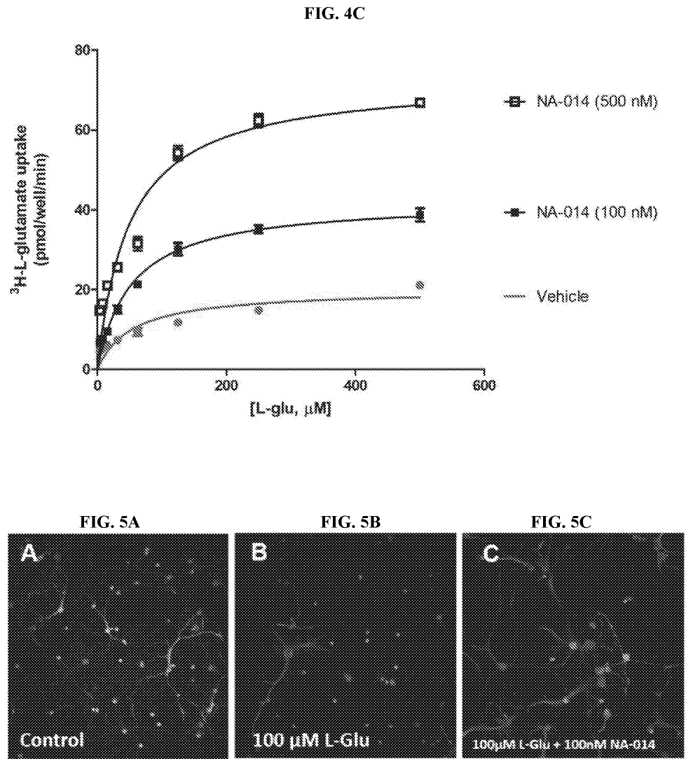

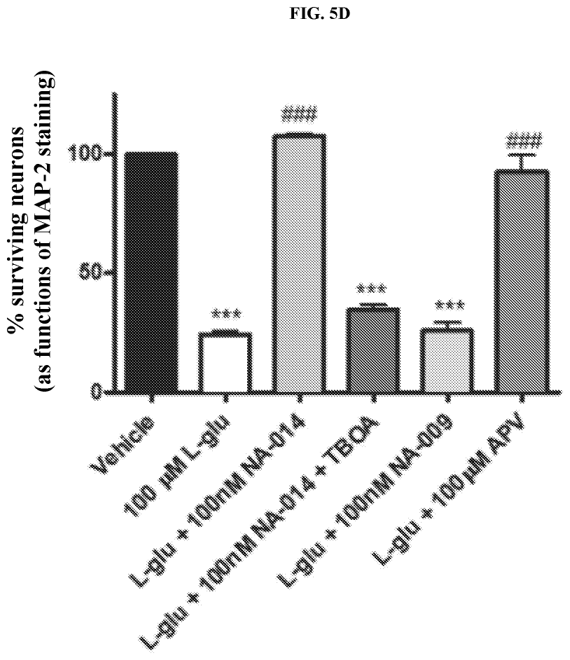

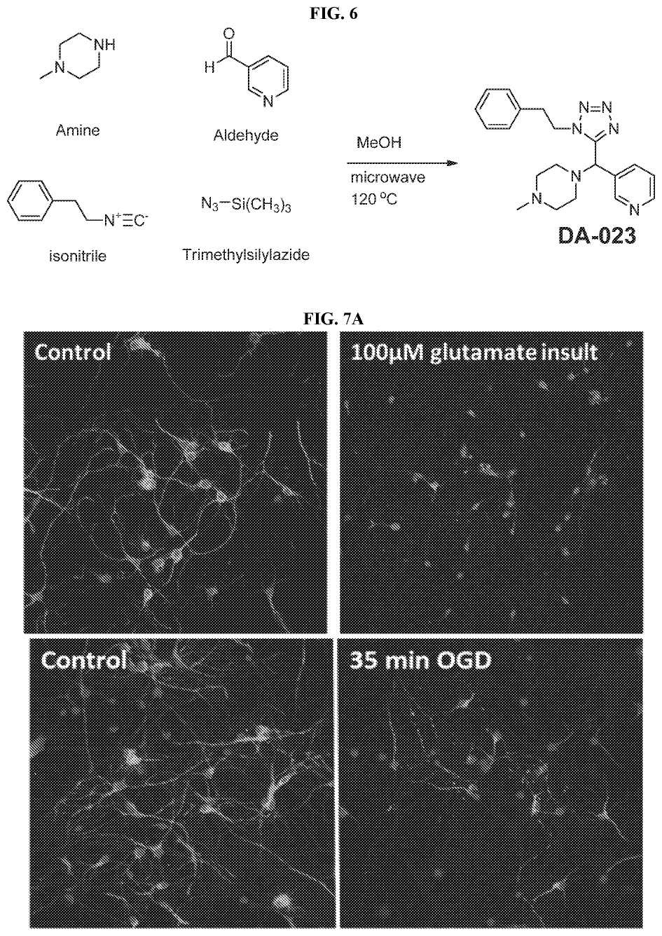

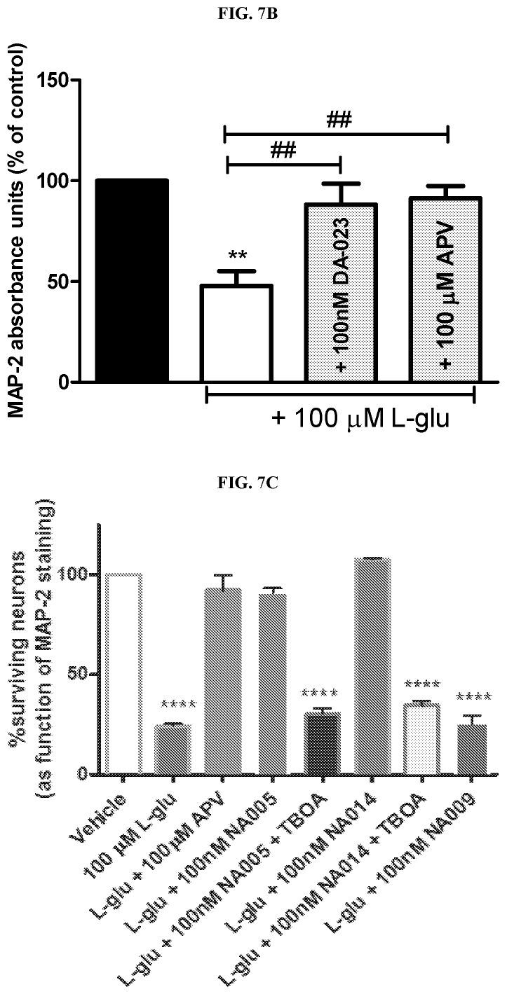

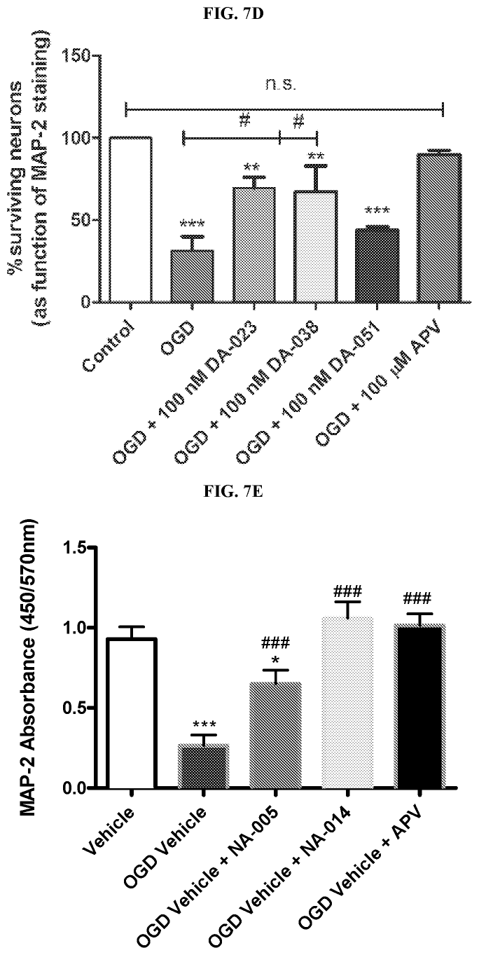

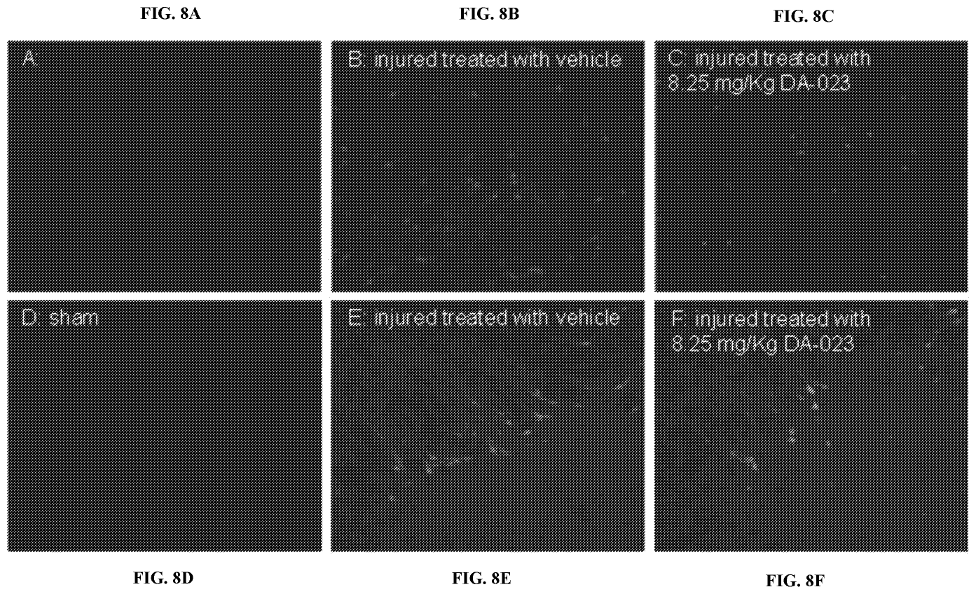

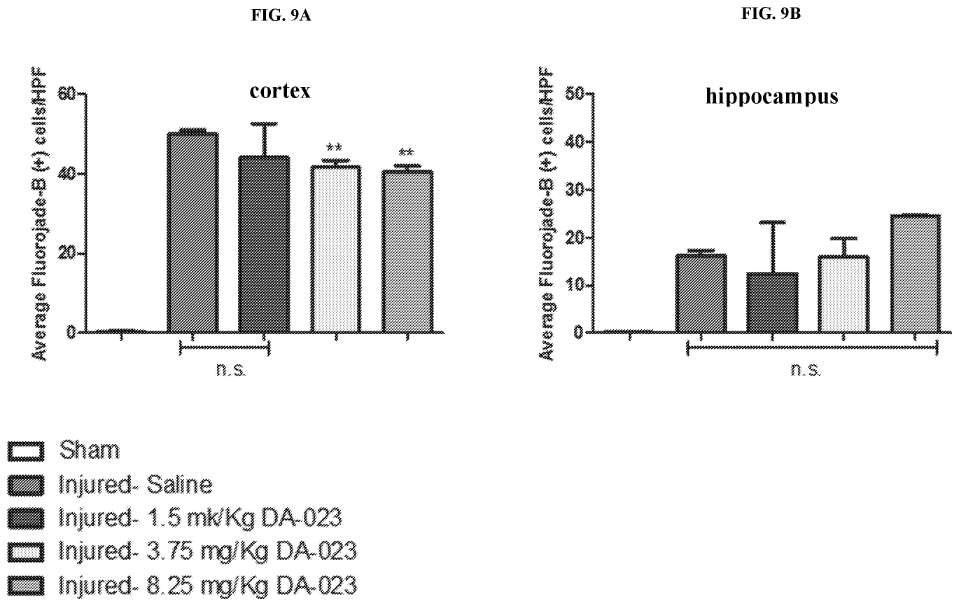

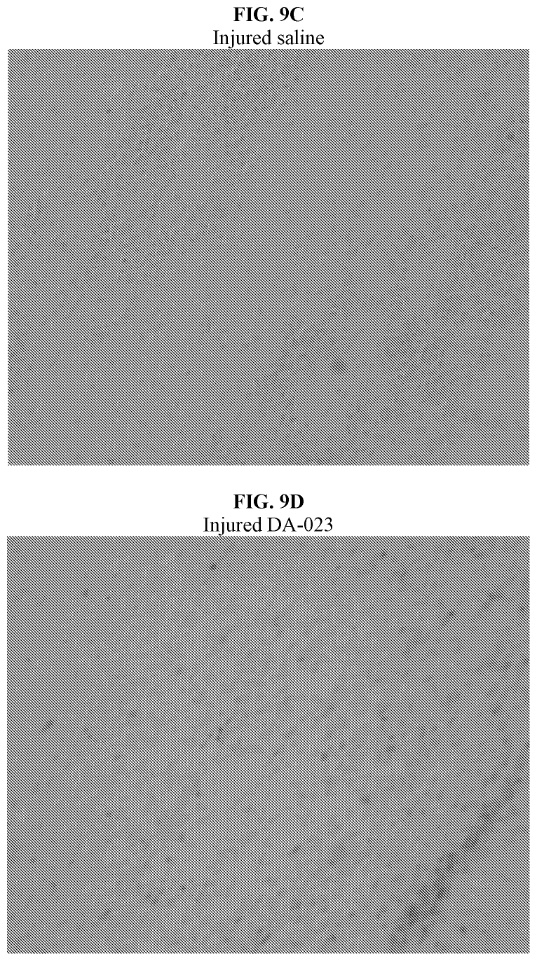

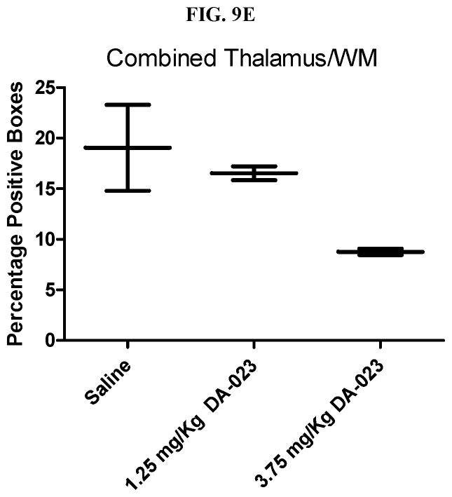

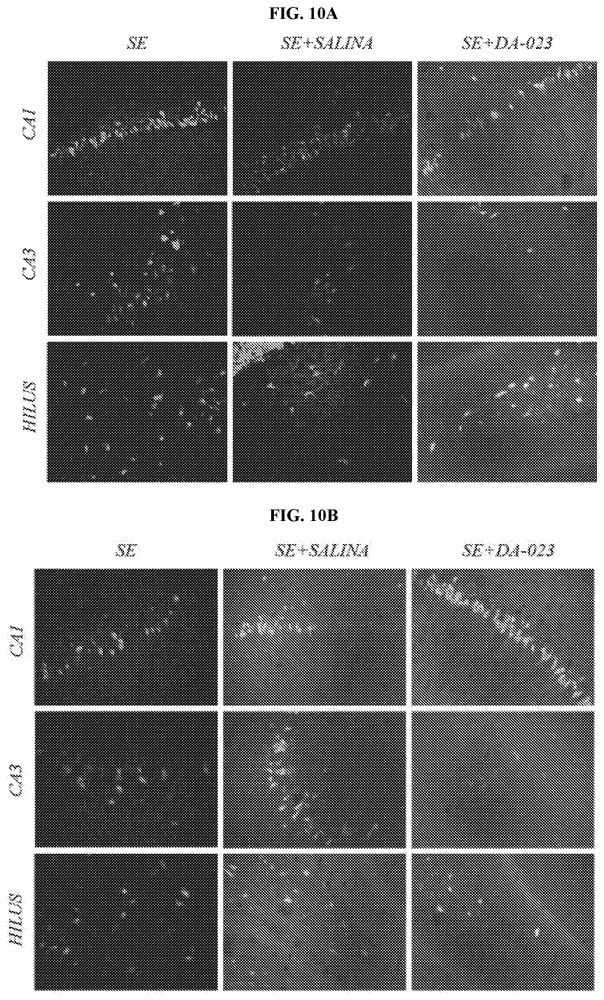

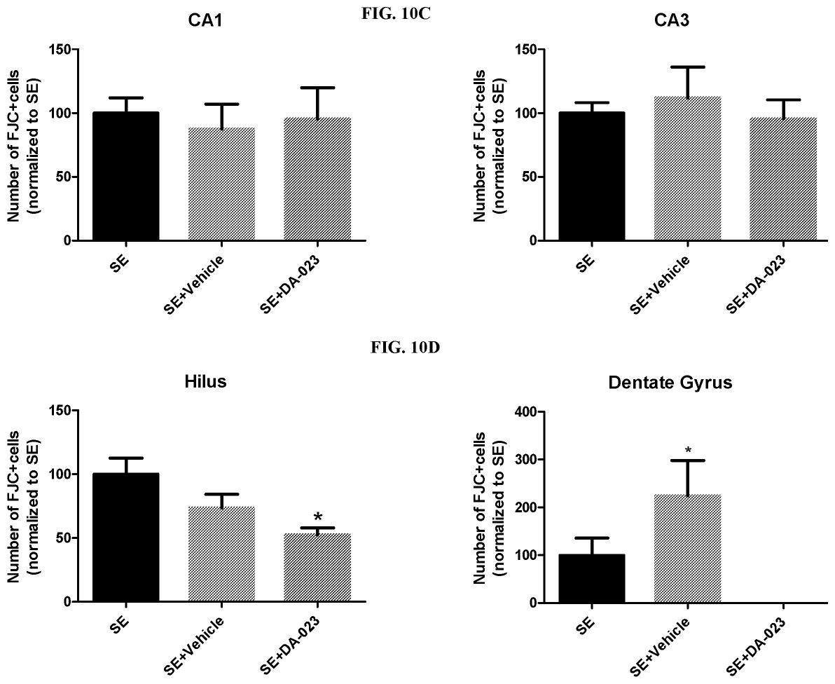

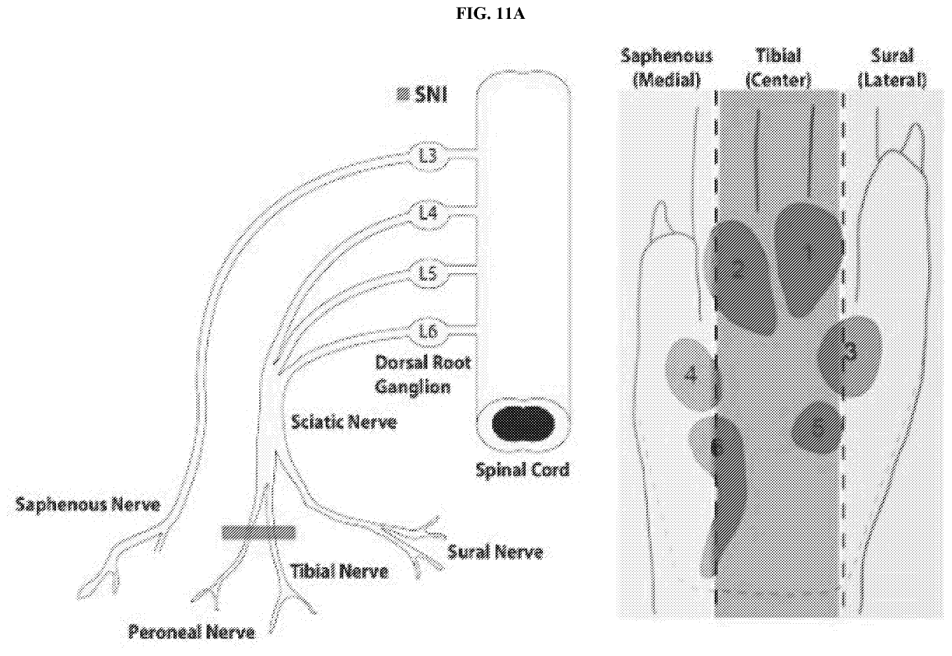

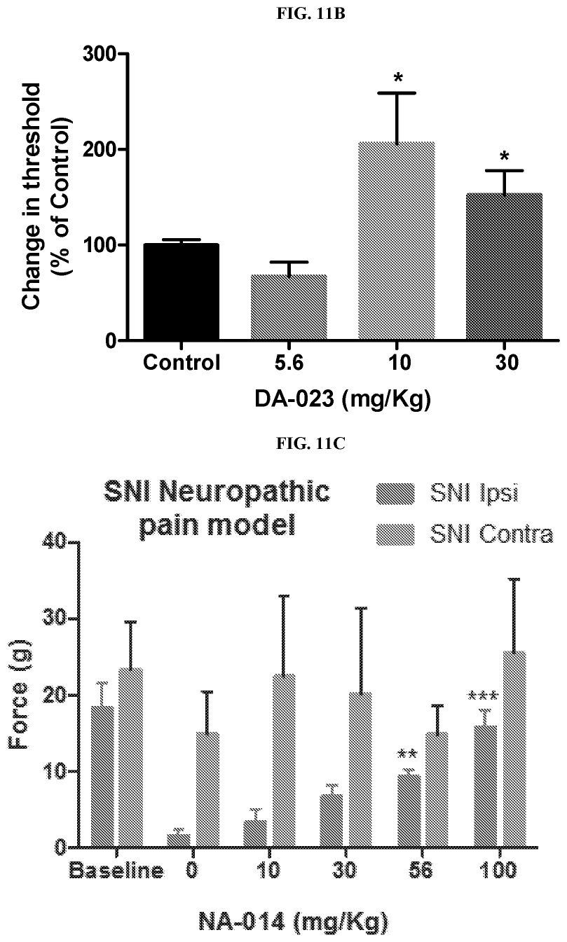

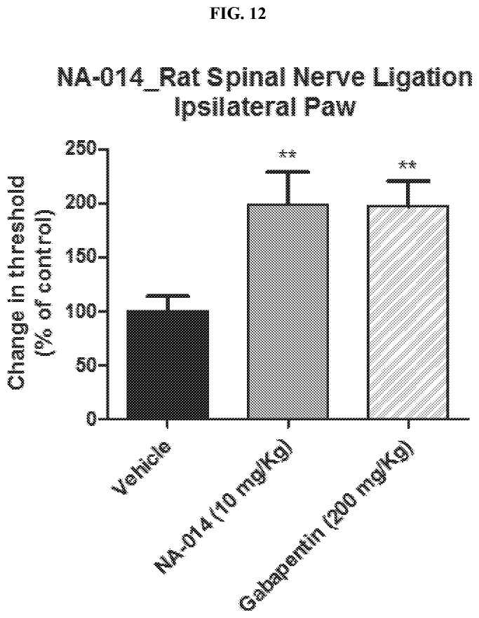

Table 5 summarizes the data obtained in studies measuring the effect of NA-014 on glutamate uptake mediated by EAAT-13 expressing COS-7 cells. The dose response curves corresponding to the data in Table 5 are in FIGS. 3A-3C.

TABLE 5

| | Vehicle | NA-014 (10 nM) | NA-014 (100 nM) | NA-014 (500 nM) |
|---|---|---|---|---|
| A. COS-7 Cells | | | | |
| Vₘₐₓ | 133 ± 13.89 | 175.4 ± 9.93 | 403.3 ± 35.82 *** | 706.4 ± 8.38 *** |
| (pmol/well/min) | | | | |
| K_{M} (μM) | 47.66 ± 28.78 | 46.92 ± 11.04 | 98.50 ± 29.68 | 67.57 ± 8.38 |
| B. Glia | | | | |
| Vₘₐₓ | 20.01 ± 1.53 | | 42.35 ± 1.44 *** | 72.76 ± 3.67 *** |
| (pmol/well/min) | | | | |
| K_{M} (μM) | 53.53 ± 13.30 | | 52.50 ± 5.82 | 49.84 ± 8.30 |

### Combination Therapies

The compounds useful within the methods described herein can be used in combination with one or more additional therapeutic agents useful for treating the diseases and disorders described herein. These additional therapeutic agents may comprise compounds that are commercially available or synthetically accessible to those skilled in the art. These additional therapeutic agents are known to treat, prevent, or reduce the symptoms, of at least one disorder described herein.

In non-limiting examples, the additional compounds comprise riluzole (6-(trifluoromethoxy)benzothiazol-2-amine) or ceftriaxone ((6R,7R)-7-{[(2Z)-2-(2-amino-1,3-thiazol-4-yl)-2-(methoxyimino)acetyl]amino}-3-{[(2-methyl-5,6-dioxo-1,2,5,6-tetrahydro-1,2,4-triazin-3-yl)thio]methyl}-8-oxo-5-thia-1-azabicyclo[4.2.0]oct-2-ene-2-carboxylic acid).

In various embodiments, a synergistic effect is observed when a compound as described herein is administered with one or more additional therapeutic agents or compounds. A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a at least one disease or disorder described as being treatable by the compounds described herein. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat a at least one disease or disorder described as being treatable by the compounds described herein in the patient. An effective amount of the therapeutic compound necessary to achieve a therapeutic effect may vary according to factors such as the state of the disease or disorder in the patient; the age, sex, and weight of the patient; and the ability of the therapeutic compound to treat a at least one disease or disorder described as being treatable by the compounds described herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms of a disease or disorder described herein in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of one or more diseases or disorders mentioned herein in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95% or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

Various embodiments of the present application can be better understood by reference to the following Examples which are offered by way of illustration. The scope of the present application is not limited to the Examples given herein.

### Materials:

Radiolabeled substrates, [³H]-glutamic acid (51.1 Ci/mmol), [³H]-dopamine (53.6 Ci/mmol), [³H]-serotonin (28.2 Ci/mmol), and [³H]-norepinephrine (14.9 Ci/mmol), were purchased from PerkinElmer (Boston, Mass., USA). DL-TBOA was purchased from Tocris (Bristol, UK). Cell culture media and supplements, including Dulbecco's modified Eagle's medium (DMEM) with glucose, Neurobasal-A, fetal bovine serum, fetal calf serum, heat-inactivated horse serum, penicillin/streptomycin, glutamine, L-glutamax, Dulbecco's phosphate-buffered saline (D-PBS), L-glutamax-1, B-27 supplement, and scintillation fluid, were obtained from Thermo Fisher Scientific (Waltham, Mass.). Transfection reagent TransIT-LT1 was from Mirus Bio LLC (Madison, Wis.). Reagents for uptake assays and nonradiolabeled substrates were purchased from Sigma-Aldrich (St. Louis, Mo.).

In some embodiments, three compounds were determined to be in vitro activators and four were found to be in vitro inhibitors of EEAT2. Further characterization of the two best ranking EAAT2 activators for efficacy, potency, and selectivity for glutamate over monoamine transporters subtypes and NMDA receptors and for efficacy in cultured astrocytes was conducted. Mutagenesis studies suggested that the EAAT2 activators interact with residues forming the interface between the trimerization and transport domains. These compounds enhance the glutamate translocation rate, with no effect on substrate interaction, suggesting an allosteric mechanism. The identification of these novel positive allosteric modulators of EAAT2 offers an innovative approach for the development of therapies based on glutamate transport enhancement.

The EAAT2 allosteric modulators described herein, in various embodiments, demonstrate a selective effect for EAAT2 on glutamate uptake. The compounds also demonstrate neuroprotective properties after glutamate and oxygen glucose deprivation insults in mixed neuron/glia cultures. In a spinal nerve ligation model, the compounds described herein, in various embodiments, show an analgesic response and also effects pain threshold in a spare nerve injury model. Additionally, the EAAT2 allosteric modulators provide neuroprotection in the hilus region of the hippocampus in a model of epilepsy, and provide neuroprotection in both neurons and axons in a model of traumatic brain injury.

### Example 1: Synthesis of DA-023 and Analogs

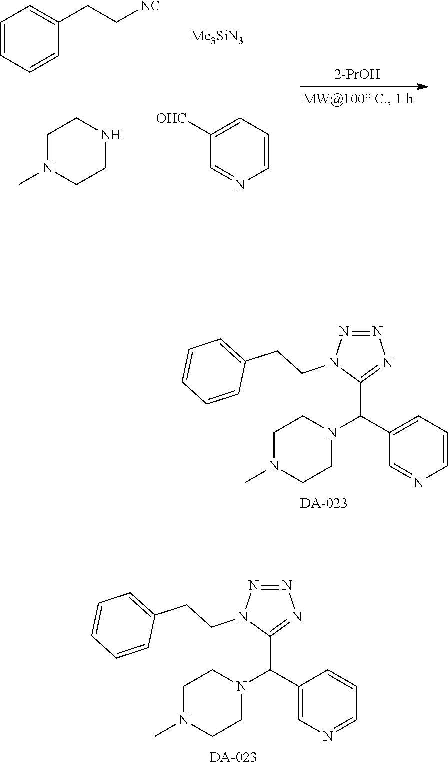

1-methyl-4-((1-phenethyl-1H-tetrazol-5-yl)(pyridin-3-yl)methyl)piperazine (DA-023): To a stirred solution of pyridine-3-carbaldehyde (100 mg, 0.93 mmol) in 3 mL of isopropanol was added 1-methylpiperazine (103 mg, 1.03 mmol), trimethylsilylazide (107 mg, 0.93 mmol) and (2-isocyanoethyl) benzene (122 mg, 0.93 mmol) were added to the reaction mixture and microwave at 100° C. for one hour. After completion of reaction as indicated by TLC reaction mixture cooled to room temperature, the solvent was evaporated under vacuum and crude residue was purified by flash chromatography using (0-15% methanol/dichloromethane) to obtain the pure compound (285 mg, 84.01% yield) as a white solid. ¹H NMR (500 MHz, Chloroform-d) δ 8.56 (tt, J=3.5, 1.6 Hz, 1H), 8.35 (t, J=2.7 Hz, 1H), 7.79 (dq, J=8.0, 1.9 Hz, 1H), 7.28 (dddd, J=9.9, 5.1, 2.6, 1.1 Hz, 5H), 7.06-6.99 (m, 2H), 4.79 (ddt, J=10.4, 7.4, 5.3 Hz, 1H), 4.68 (dtd, J=13.5, 6.2, 2.9 Hz, 1H), 4.28 (d, J=2.7 Hz, 1H), 3.21 (td, J=7.0, 5.8, 3.6 Hz, 2H), 2.40 (s, 2H), 2.33-2.22 (m, 9H). MS (ESI): m/z 364.3 [M+1]⁺; HRMS (ESI m z) for C₂₀H₂₆N₇, calcd 364.22442, found 364.22399 [M+1]⁺.

### Example 2: Synthesis of NA-014

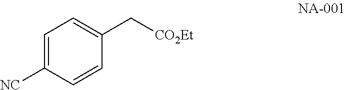 [SMILES: CCOC(=O)Cc1ccc(C#N)cc1]

### Ethyl 2-(4-cyanophenyl)acetate

Ethyl 2-(4-cyanophenyl)acetate (NA-001): To a solution of 4-cyanophenylacetic acid (40 g) in 250 mL ethanol was added catalytic amount of sulfuric acid (2 g). The reaction mixture refluxed for 24 h, then cooled to room temperature. Sat. NaHCO₃ was added at 0° C. The solvent was evaporated and crude residue was diluted water and extracted into ethyl acetate. The EtOAC phase was concentrated and the residue was crystallized in EtOAc/hexane (1:2) to give a white solid 45 g. MS (ESI): m/z 190.1 [M+1]⁺.

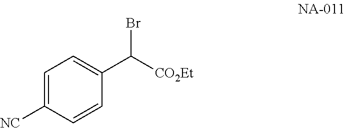 [SMILES: CCOC(=O)C(Br)c1ccc(C#N)cc1]

### Ethyl 2-bromo-2-(4-cyanophenyl)acetate

Ethyl 2-bromo-2-(4-cyanophenyl)acetate (NA-011): To a solution of ethyl 2-(4-cyanophenyl)acetate (18.9 g, 100 mmol) in 250 mL CCl₄ at RT was added (PhCOO)₂ (2.42 g, 10 mmol) and NBS (18.25 g, 102.5 mmol). The reaction mixture was heated to reflux for 3 h and additional NBS (1 g) was added. After additional 5 h, the reaction was cooled to RT and filtered. The filter was diluted with DCM and washed with water, concentrated to give the crude product 27.92 g, (brown oil, slightly less polar than compound NA-001 at TLC using 4:1 of hexane/EtOAc), which was directly used for the next step without purification. MS (ESI): m/z 267.9 [M+1]⁺.

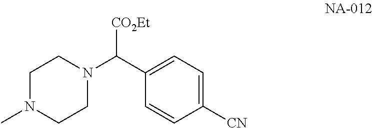 [SMILES: CCOC(=O)C(c1ccc(C#N)cc1)N1CCN(C)CC1]

### Ethyl 2-(4-cyanophenyl)-2-(4-methylpiperazin-1-yl)acetate

Ethyl 2-(4-cyanophenyl)-2-(4-methylpiperazin-1-yl)acetate (NA-012): The crude ethyl 2-bromo-2-(4-cyanophenyl)acetate (100 mmol) was dissolved in 200 mL CH₃CN. N-methyl piperazine (10 g) and triethylamine (14 mL) were added and reaction mixture was stirred at RT for 4 h. The reaction was concentrated, dissolved in EtOAc, washed with sat. NaHCO₃ and water, concentrated, purified by flash column (2:1-1:2/Hexane:EtOAc) to give a light brown oil 23 g. MS (ESI): m/z 288.2 [M+1]⁺.

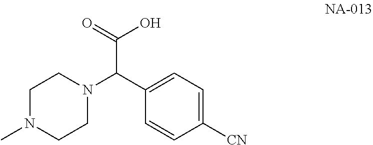 [SMILES: CN1CCN(C(C(=O)O)c2ccc(C#N)cc2)CC1]

### 2-(4-cyanophenyl)-2-(4-methylpiperazin-1-yl)acetic acid

2-(4-cyanophenyl)-2-(4-methylpiperazin-1-yl)acetic acid (NA-013): To a solution of ethyl 2-(4-cyanophenyl)-2-(4-methylpiperazin-1-yl)acetate (20 g, 69.69 mmol) dissolved 300 mL of methanol:tetrahydrofuran (1:1) was added 150 mL of 1N lithium hydroxide at 0° C. The reaction mixture was stirred at room temperature for 12. After completion of reaction, 40 mL of 4N hydrochloric acid was added at 0° C. The solvent was evaporated (toluene needed) to dry to give a white solid, which was directly used for the next step without purification. MS (ESI): m/z 260.2 [M+1]⁺.

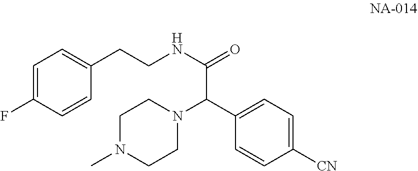 [SMILES: CN1CCN(C(C(=O)NCCc2ccc(F)cc2)c2ccc(C#N)cc2)CC1]

### 2-(4-cyanophenyl)-N-(4-fluorophenethyl)-2-(4-methylpiperazin-1-yl)acetamide

2-(4-cyanophenyl)-N-(4-fluorophenethyl)-2-(4-methylpiperazin-1-yl)acetamide (NA-014): To a stirred solution of crude 2-(4-cyanophenyl)-2-(4-methylpiperazin-1-yl)acetic acid (NA-013, 69.69 mmol) in DMF (120 mL), 4-Fluorophenylethylamine (10 g), DIPEA (13 mL) and HATU (27 g) were added at 0° C. The resulting mixture was stirred at room temperature for 16 h and then the reaction mixture was quenched with ice cold water and extracted with EtOAc. The organic layer was evaporated under vacuum and the resulting product was purified by flash column chromatography employing 2:1/hexane:EtOAc, 1:1/hexane:acetone and 20:1:0.1/DCM:MeOH:NH₄OH as an eluents to give a crude solid (˜90% purity). The crude solid was further purified by crystallization in hexane:acetone (4:1) to give a white solid 14.75 g. Additional ˜15 g of impure product (50%) was kept as oil. MS (ESI): m/z 381.8 [M+1]⁺.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following exemplary embodiments are provided, the numbering of which is not to be construed as designating levels of importance:

Embodiment 1 provides a compound of Formula I or II, or an enantiomer, diastereoisomer, tautomer, salt or solvate thereof, having the formula:

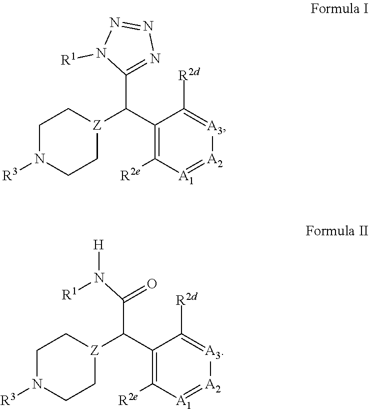

wherein:

R¹ is selected from the group consisting of —C₁-C₆ alkyl, —C₁-C₆ heteroalkyl, —(C₀-C₃ alkyl)-(C₃-C₆ cycloalkyl), —(C₀-C₃ alkyl)-(C₄-C₁₀ heterocyclyl), —(C₀-C₃ alkyl)-(C₆-C₁₀ aryl) and —(C₀-C₃ alkyl)-(C₅-C₁₀ heteroaryl), wherein the alkyl, heteroalkyl, cycloalkyl, heterocyclyl, aryl and heteroaryl groups are independently optionally substituted;
A₁ is N or CR^{2a};
A₂ is N or CR^{2b};
A₃ is N or CR^{2c};

each occurrence of R^{2a}, R^{2b}, R^{2c}, R^{2d}, and R^{2e} is independently selected from the group consisting of H, tritium (³H), —C₁-C₆ alkyl, —OH, —C₁-C₆ alkoxy, halogen, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂;

R³ is selected from the group consisting of —(C═O)₀₋₁(C₁-C₆ alkyl), —(C═O)₀₋₁(C₁-C₆ heteroalkyl), —(C═O)₀₋₁(C₀-C₃ alkyl)-(C₃-C₆ cycloalkyl), —(C═O)₀₋₁(C₀-C₃ alkyl)-(C₄-C₁₀ heterocyclyl), —(C═O)₀₋₁(C₀-C₃ alkyl)-(C₆-C₁₀ aryl), —(C═O)₀₋₁(C₀-C₃ alkyl)-(C₅-C₁₀ heteroaryl), —(SO₂)₀₋₁(C₁-C₆ alkyl), —(SO₂)₀₋₁(C₁-C₆ heteroalkyl), —(SO₂)₀₋₁(C₀-C₃ alkyl)-(C₃-C₆ cycloalkyl), —(SO₂)₀₋₁(C₀-C₃ alkyl)-(C₄-C₁₀ heterocyclyl), —(SO₂)₀₋₁(C₀-C₃ alkyl)-(C₆-C₁₀ aryl) and —(SO₂)₀₋₁(C₀-C₃ alkyl)-(C₅-C₁₀ heteroaryl), wherein the alkyl, heteroalkyl, cycloalkyl, heterocyclyl, aryl and heteroaryl groups are independently optionally substituted; and

Z is N or CH.

Embodiment 2 provides the compound of embodiment 1, which is of Formula I-1 or I-2:

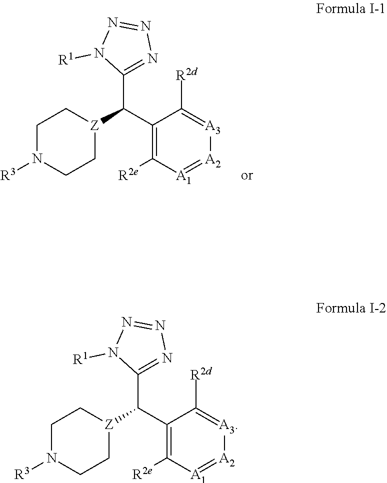

Embodiment 3 provides the compound of any one of embodiments 1-2, which is of Formula II-1 or II-2:

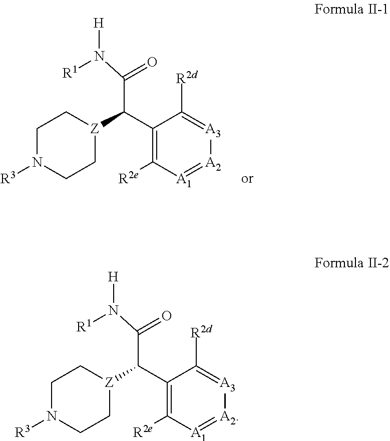

Embodiment 4 provides the compound of any one of embodiments 1-3, wherein one or more hydrogen atoms in R¹ are independently replaced with deuterium (2H) and/or tritium (3H).

Embodiment 5 provides the compound of any one of embodiments 1-4, wherein Z is N.

Embodiment 6 provides the compound of any one of embodiments 1-5, wherein only one of A₁, A₂, and A₃ is N.

Embodiment 7 provides the compound of any one of embodiments 1-6, wherein A₁ is CR^{2a}, A₂ is CR^{2b}, and A₃ is CR^{2e}.

Embodiment 8 provides the compound of any one of embodiments 1-7, wherein R¹ is selected from —(C₀-C₃ alkyl)-(C₃-C₆ cycloalkyl), —(C₀-C₃ alkyl)-(C₄-C₁₀ heterocyclyl), —(C₀-C₃ alkyl)-(C₆-C₁₀ aryl) and —(C₀-C₃ alkyl)-(C₅-C₁₀ heteroaryl).

Embodiment 9 provides the compound of any one of embodiments 1-8, wherein R¹ is selected from —(C₀-C₃ alkyl)-(C₃-C₆ cycloalkyl) and —(C₀-C₃ alkyl)-(C₆-C₁₀ aryl).

Embodiment 10 provides the compound of any one of embodiments 1-9, wherein R¹ is —(C₀-C₃ alkyl)-phenyl, and wherein the phenyl group is optionally substituted by 1 to 5 groups selected from the group consisting of tritium (³H), —C₁-C₆ alkyl, —OH, —C₁-C₆ alkoxy, halogen, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂.

Embodiment 11 provides the compound of any one of embodiments 1-10, wherein each occurrence of R^{2a}, R^{2b}, R^{2c}, R^{2d}, and R^{2e} is independently selected from the group consisting of H, tritium (³H), —OH, —C₁-C₆ alkoxy, halogen, trifluoromethyl, and —C≡N.

Embodiment 12 provides the compound of any one of embodiments 1-11, wherein R³ is selected from the group consisting of —(C═O)₀₋₁(C₁-C₆ alkyl), —(C═O)₀₋₁(C₁-C₆ heteroalkyl), and —(C═O)₀₋₁(C₀-C₃ alkyl)-(C₃-C₆ cycloalkyl).

Embodiment 13 provides the compound of any one of embodiments 1-12, wherein R³ is methyl or cyclohexyl.

Embodiment 14 provides the compound of any one of embodiments 1-13, wherein the compound is selected from the group consisting of:

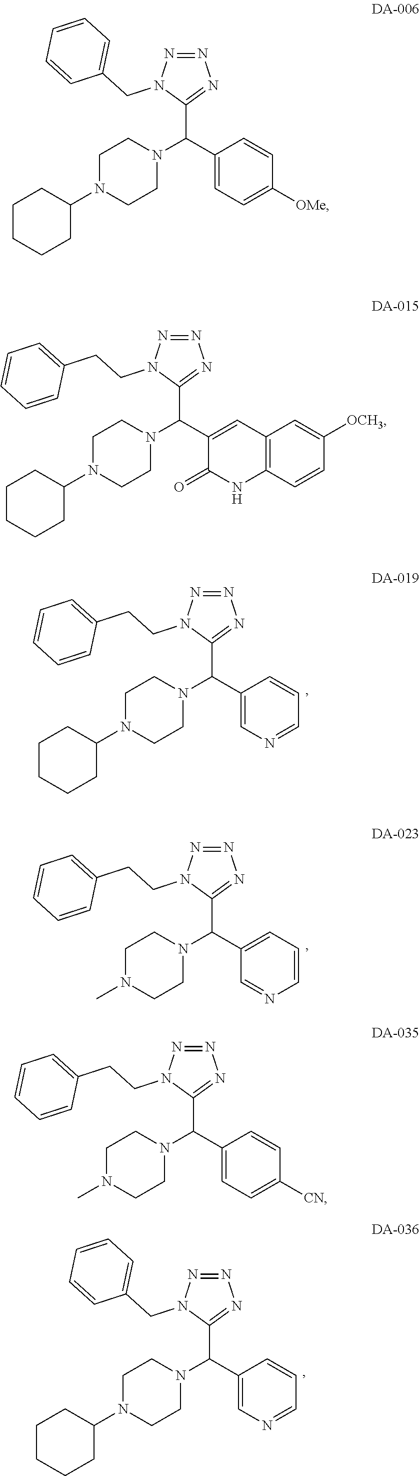

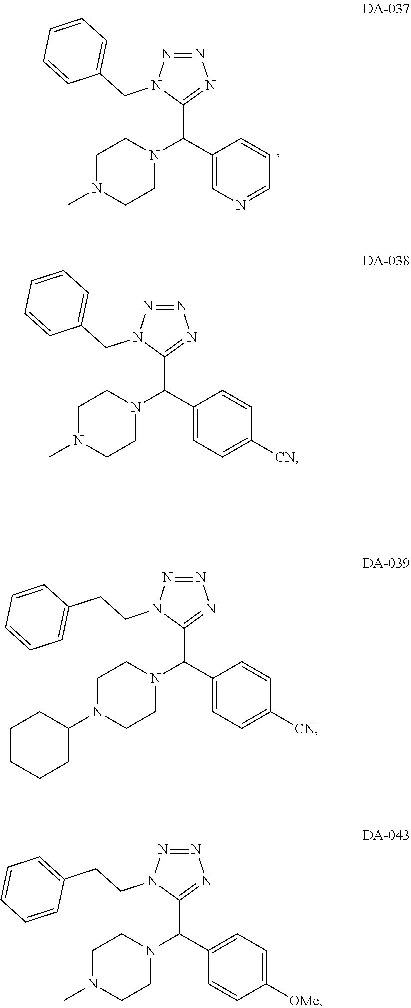 [SMILES: CN1CCN(C(c2cccnc2)c2nnnn2Cc2ccccc2)CC1 ; "," ; CN1CCN(C(c2ccc(C#N)cc2)c2nnnn2Cc2ccccc2)CC1 ; N#Cc1ccc(C(c2nnnn2CCc2ccccc2)N2CCN(C3CCCCC3)CC2)cc1 ; COc1ccc(C(c2nnnn2CCc2ccccc2)N2CCN(C)CC2)cc1]

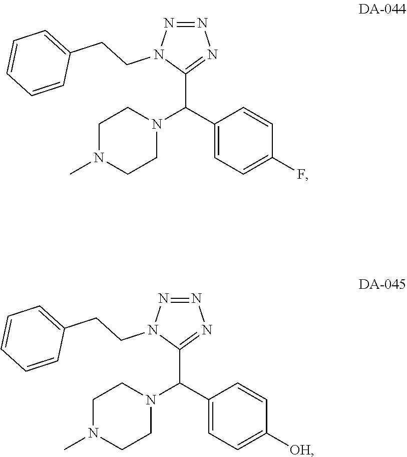 [SMILES: CN1CCN(C(c2ccc(F)cc2)c2nnnn2CCc2ccccc2)CC1 ; CN1CCN(C(c2ccc(O)cc2)c2nnnn2CCc2ccccc2)CC1]

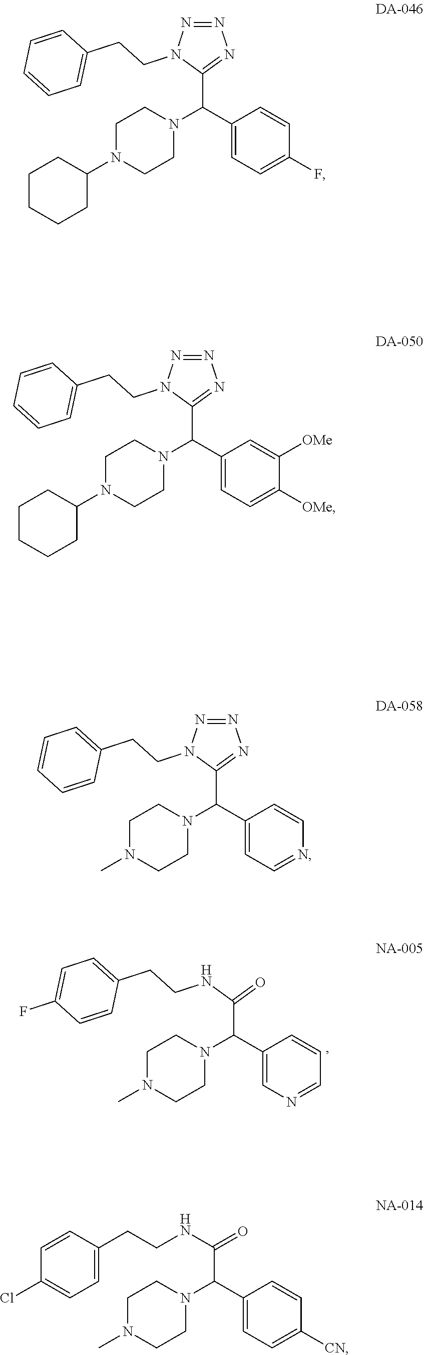 [SMILES: Fc1ccc(C(c2nnnn2CCc2ccccc2)N2CCN(C3CCCCC3)CC2)cc1 ; COc1ccc(C(c2nnnn2CCc2ccccc2)N2CCN(C3CCCCC3)CC2)cc1OC ; CN1CCN(C(c2ccncc2)c2nnnn2CCc2ccccc2)CC1 ; CN1CCN(C(C(=O)NCCc2ccc(F)cc2)c2cccnc2)CC1 ; "," ; CN1CCN(C(C(=O)NCCc2ccc(Cl)cc2)c2ccc(C#N)cc2)CC1]

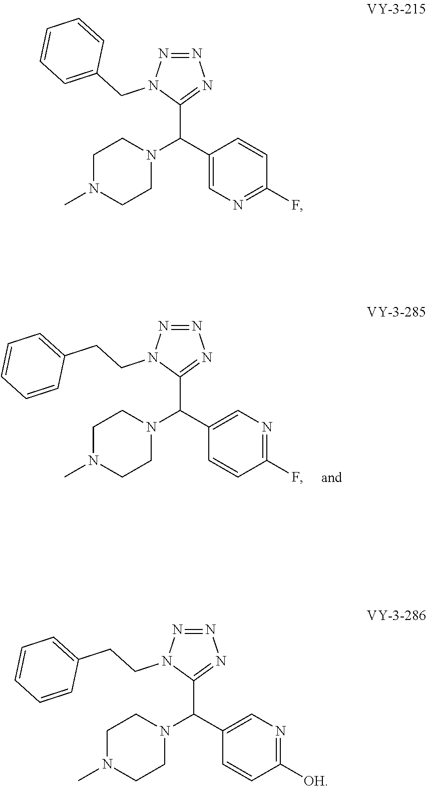 [SMILES: CN1CCN(C(c2ccc(F)nc2)c2nnnn2Cc2ccccc2)CC1 ; CN1CCN(C(c2ccc(F)nc2)c2nnnn2CCc2ccccc2)CC1 ; "and" ; CN1CCN(C(c2ccc(O)nc2)c2nnnn2CCc2ccccc2)CC1]

Embodiment 15 provides a method of treating, preventing, and/or ameliorating a disease or disorder that is caused, induced or characterized by abnormal reduction in glutamate transporter activity or abnormal increase in extracellular CNS glutamate concentration in a subject, the method comprising administering to the subject a therapeutically effective amount of a compound of any one of embodiments 1-14.

Embodiment 16 provides the method of embodiment 15, wherein the disease or disorder is at least one selected from the group consisting of ischemia, seizure, traumatic brain injury, stroke, epilepsy, schizophrenia, and neurodegenerative diseases or disorders.

Embodiment 17 provides the method of any one of embodiments 15-16, wherein the disease or disorder is at least one selected from the group consisting of ischemia, seizure, traumatic brain injury, stroke, epilepsy, schizophrenia, Parkinson's disease, Alzheimer's disease, Huntington's disease, amyotrophic lateral sclerosis (ALS), and ALS-parkinsonism dementia complex, HIV-associated neurocognitive disorder (HAND), neuropathic pain, mental health disorders and drug abuse/relapse.

Embodiment 18 provides the method of any one of embodiments 15-17, wherein the compound activates, stimulates and/or upregulates the activity of a glutamate transporter in the subject.

Embodiment 19 provides the method of any one of embodiments 15-18, wherein the transporter comprises EAAT2.

Embodiment 20 provides the method of any one of embodiments 15-19, wherein administration of the compound regulates extracellular glutamate concentrations in the subject.

Embodiment 21 provides the method of any one of embodiments 15-20, wherein administration of the compound increases, induces or upregulates removal of glutamate from the neuronal synaptic cleft into neuroglia and neurons of the subject.

Embodiment 22 provides the method of any one of embodiments 15-21, wherein administration of the compound inhibits glutamate transport in the subject.

Embodiment 23 provides the method of any one of embodiments 15-22, wherein the compound is administered to the subject as part of a pharmaceutical composition.

Embodiment 24 provides the method of any one of embodiments 15-23, wherein the subject is further administered at least one additional therapeutic agent.

Embodiment 25 provides the method of any one of embodiments 15-24, wherein the subject is a mammal.

Embodiment 26 provides the method of any one of embodiments 15-25, wherein the mammal is human.

Embodiment 27 provides the method of any one of embodiments 15-26, wherein the compound is selected from the group consisting of

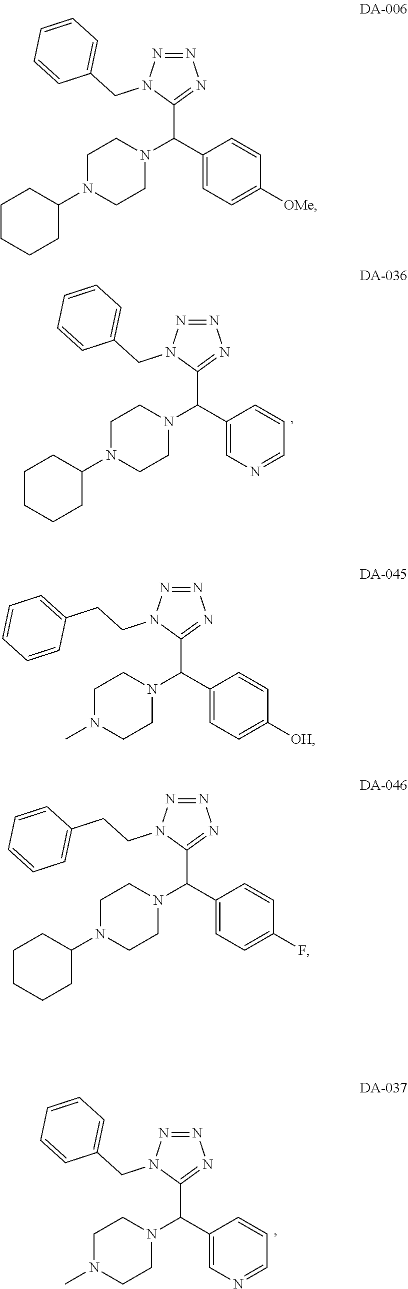

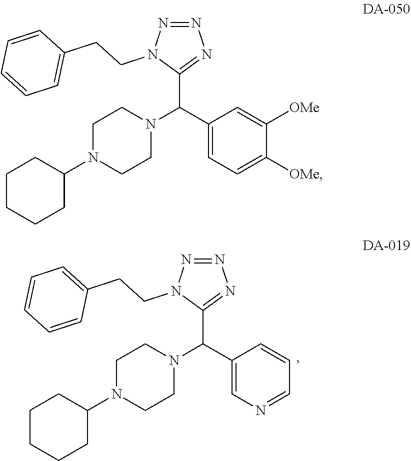 [SMILES: COc1ccc(C(c2nnnn2CCc2ccccc2)N2CCN(C3CCCCC3)CC2)cc1OC ; c1ccc(CCn2nnnc2C(c2cccnc2)N2CCN(C3CCCCC3)CC2)cc1 ; ","]

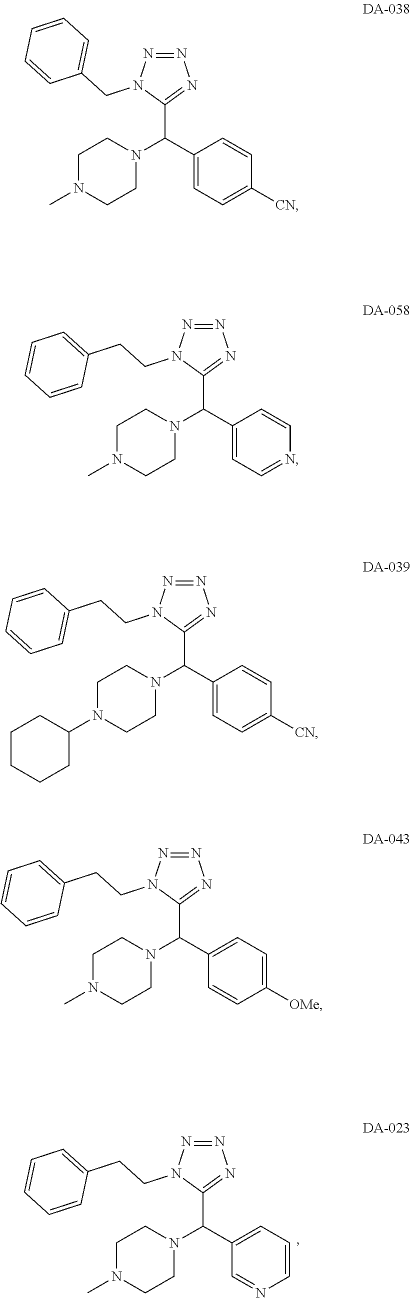 [SMILES: CN1CCN(C(c2ccc(C#N)cc2)c2nnnn2Cc2ccccc2)CC1 ; CN1CCN(C(c2ccncc2)c2nnnn2CCc2ccccc2)CC1 ; N#Cc1ccc(C(c2nnnn2CCc2ccccc2)N2CCN(C3CCCCC3)CC2)cc1 ; COc1ccc(C(c2nnnn2CCc2ccccc2)N2CCN(C)CC2)cc1 ; CN1CCN(C(c2cccnc2)c2nnnn2CCc2ccccc2)CC1 ; ","]

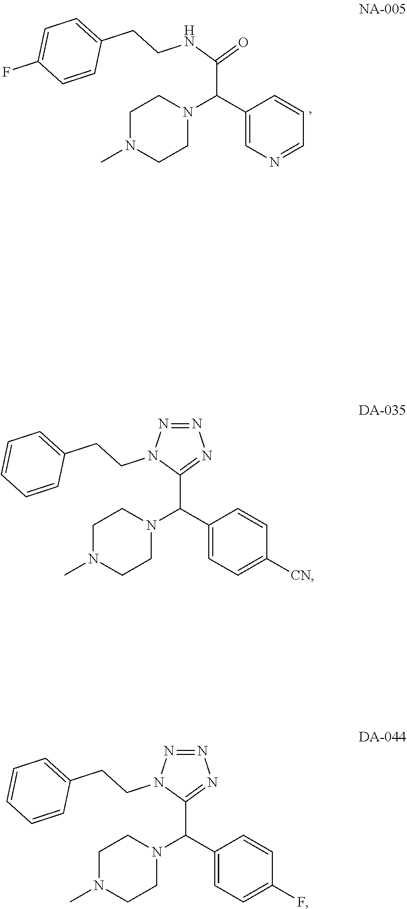 [SMILES: CN1CCN(C(C(=O)NCCc2ccc(F)cc2)c2cccnc2)CC1 ; "," ; CN1CCN(C(c2ccc(C#N)cc2)c2nnnn2CCc2ccccc2)CC1 ; CN1CCN(C(c2ccc(F)cc2)c2nnnn2CCc2ccccc2)CC1]

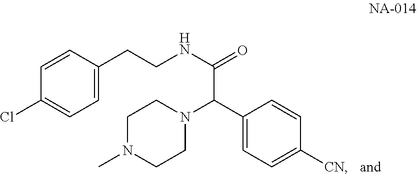 [SMILES: CN1CCN(C(C(=O)NCCc2ccc(Cl)cc2)c2ccc(C#N)cc2)CC1 ; "and"]

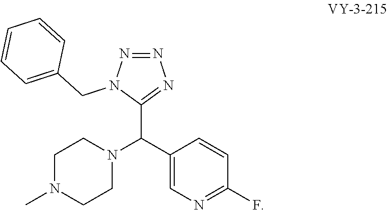 [SMILES: CN1CCN(C(c2ccc(F)nc2)c2nnnn2Cc2ccccc2)CC1]

1. A compound of Formula I or II, or an enantiomer, diastereoisomer, tautomer, salt or solvate thereof, having the formula: 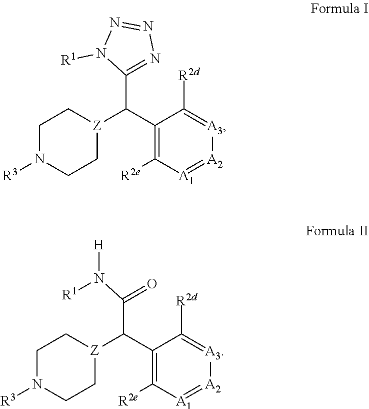
wherein:
R¹ is selected from the group consisting of —C₁-C₆ alkyl, —C₁-C₆ heteroalkyl, —(C₀-C₃ alkyl)-(C₃-C₆ cycloalkyl), —(C₀-C₃ alkyl)-(C₄-C₁₀ heterocyclyl), —(C₀-C₃ alkyl)-(C₆-C₁₀ aryl) and —(C₀-C₃ alkyl)-(C₅-C₁₀ heteroaryl),
wherein the alkyl, heteroalkyl, cycloalkyl, heterocyclyl, aryl and heteroaryl groups are independently optionally substituted;
A₁ is N or CR^{2a};
A₂ is N or CR^{2b};
A₃ is N or CR^{2c};
each occurrence of R^{2a}, R^{2b}, R^{2c}, R^{2d}, and R^{2e} is independently selected from the group consisting of H, tritium (³H), —C₁-C₆ alkyl, —OH, —C₁-C₆ alkoxy, halogen, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂;
R³ is selected from the group consisting of —(C═O)₀₋₁(C₁-C₆ alkyl), —(C═O)₀₋₁(C₁-C₆ heteroalkyl), —(C═O)₀₋₁(C₀-C₃ alkyl)-(C₃-C₆ cycloalkyl), —(C═O)₀₋₁(C₀-C₃ alkyl)-(C₄-C₁₀ heterocyclyl), —(C═O)₀₋₁(C₀-C₃ alkyl)-(C₆-C₁₀ aryl), —(C═O)₀₋₁(C₀-C₃ alkyl)-(C₅-C₁₀ heteroaryl), —(SO₂)₀₋₁(C₁-C₆ alkyl), —(SO₂)₀₋₁(C₁-C₆ heteroalkyl), —(SO₂)₀₋₁(C₀-C₃ alkyl)-(C₃-C₆ cycloalkyl), —(SO₂)₀₋₁(C₀-C₃ alkyl)-(C₄-C₁₀ heterocyclyl), —(SO₂)₀₋₁(C₀-C₃ alkyl)-(C₆-C₁₀ aryl) and —(SO₂)₀₋₁(C₀-C₃ alkyl)-(C₅-C₁₀ heteroaryl),
wherein the alkyl, heteroalkyl, cycloalkyl, heterocyclyl, aryl and heteroaryl groups are independently optionally substituted; and
Z is N or CH.

2. The compound of claim 1, which is of Formula I-1 or I-2:

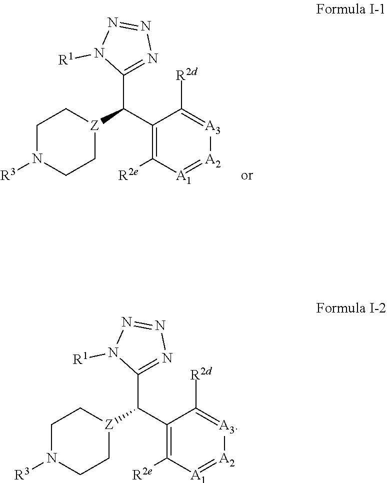

3. The compound of claim 1, which is of Formula II-1 or II-2:

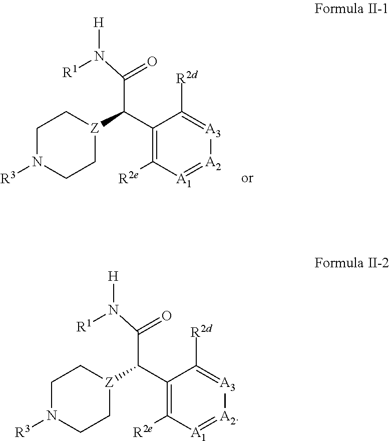

4. The compound of claim 1, wherein one or more hydrogen atoms in R¹ are independently replaced with deuterium (2H) and/or tritium (3H).

5. The compound of claim 1, wherein Z is N.

6. The compound of claim 1, wherein only one of A₁, A₂, and A₃ is N.

7. The compound of claim 1, wherein A₁ is CR^{2a}, A₂ is CR^{2b}, and A₃ is CR^{2c}

8. The compound of claim 1, wherein R¹ is selected from —(C₀-C₃ alkyl)-(C₃-C₆ cycloalkyl), —(C₀-C₃ alkyl)-(C₄-C₁₀ heterocyclyl), —(C₀-C₃ alkyl)-(C₆-C₁₀ aryl) and —(C₀-C₃ alkyl)-(C₅-C₁₀ heteroaryl).

9. The compound of claim 8, wherein R¹ is selected from —(C₀-C₃ alkyl)-(C₃-C₆ cycloalkyl) and —(C₀-C₃ alkyl)-(C₆-C₁₀ aryl).

10. The compound of claim 9, wherein R¹ is —(C₀-C₃ alkyl)-phenyl, and wherein the phenyl group is optionally substituted by 1 to 5 groups selected from the group consisting of tritium (³H), —C₁-C₆ alkyl, —OH, —C₁-C₆ alkoxy, halogen, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂.

11. The compound of claim 1, wherein each occurrence of R², R^{2b}, R^{2c}, R^{2d}, and R^{2e} is independently selected from the group consisting of H, tritium (³H), —OH, —C₁-C₆ alkoxy, halogen, trifluoromethyl, and —C≡N.

12. The compound of claim 1, wherein R³ is selected from the group consisting of —(C═O)₀₋₁(C₁-C₆ alkyl), —(C═O)₀₋₁(C₁-C₆ heteroalkyl), and —(C═O)₀₋₁(C₀-C₃ alkyl)-(C₃-C₆ cycloalkyl).

13. The compound of claim 1, wherein R³ is methyl or cyclohexyl.

14. The compound of claim 1, wherein the compound is selected from the group consisting of:

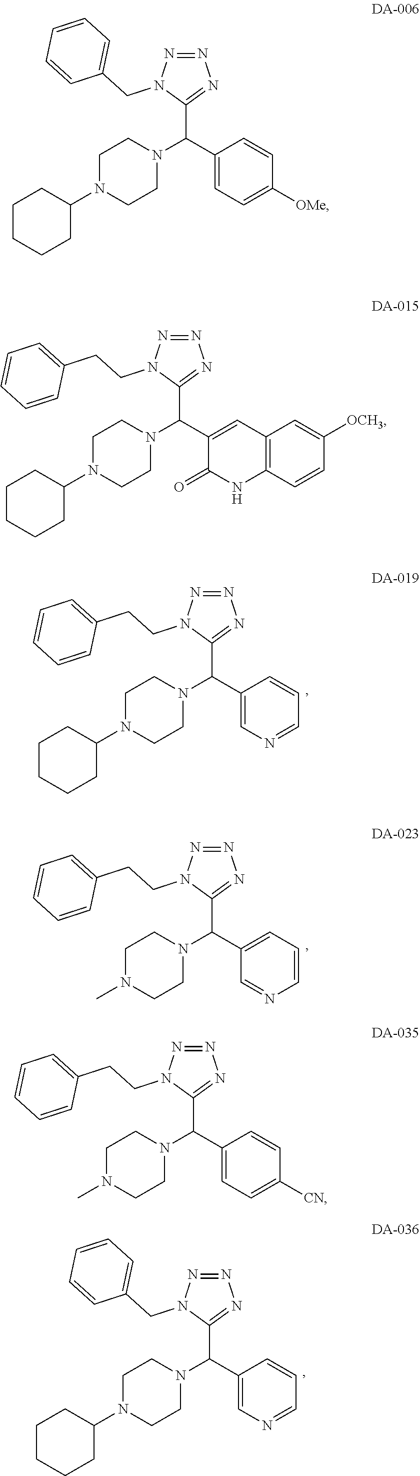

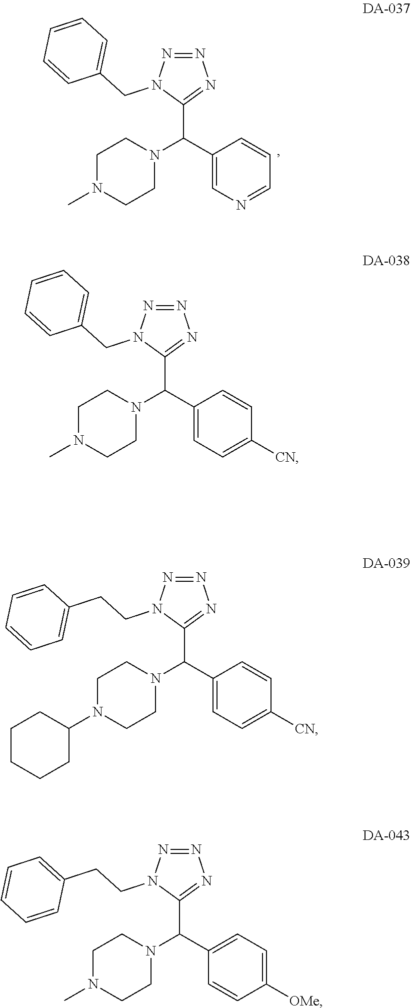 [SMILES: CN1CCN(C(c2cccnc2)c2nnnn2Cc2ccccc2)CC1 ; "," ; CN1CCN(C(c2ccc(C#N)cc2)c2nnnn2Cc2ccccc2)CC1 ; N#Cc1ccc(C(c2nnnn2CCc2ccccc2)N2CCN(C3CCCCC3)CC2)cc1 ; COc1ccc(C(c2nnnn2CCc2ccccc2)N2CCN(C)CC2)cc1]

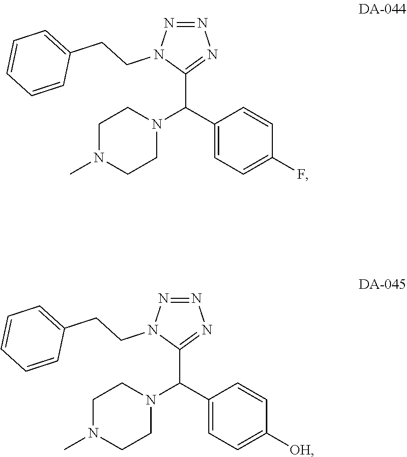 [SMILES: CN1CCN(C(c2ccc(F)cc2)c2nnnn2CCc2ccccc2)CC1 ; CN1CCN(C(c2ccc(O)cc2)c2nnnn2CCc2ccccc2)CC1]

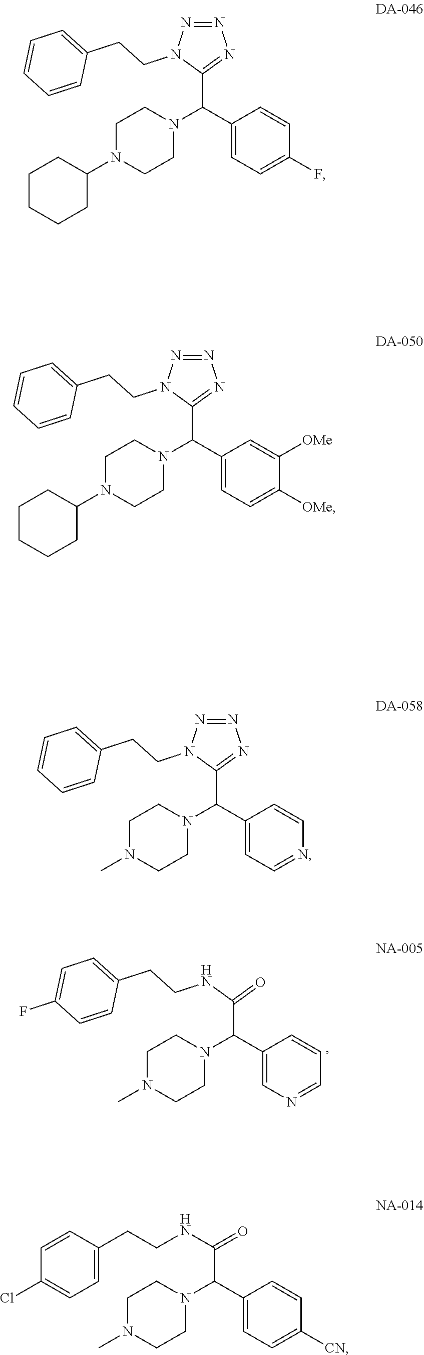 [SMILES: Fc1ccc(C(c2nnnn2CCc2ccccc2)N2CCN(C3CCCCC3)CC2)cc1 ; COc1ccc(C(c2nnnn2CCc2ccccc2)N2CCN(C3CCCCC3)CC2)cc1OC ; CN1CCN(C(c2ccncc2)c2nnnn2CCc2ccccc2)CC1 ; CN1CCN(C(C(=O)NCCc2ccc(F)cc2)c2cccnc2)CC1 ; "," ; CN1CCN(C(C(=O)NCCc2ccc(Cl)cc2)c2ccc(C#N)cc2)CC1]

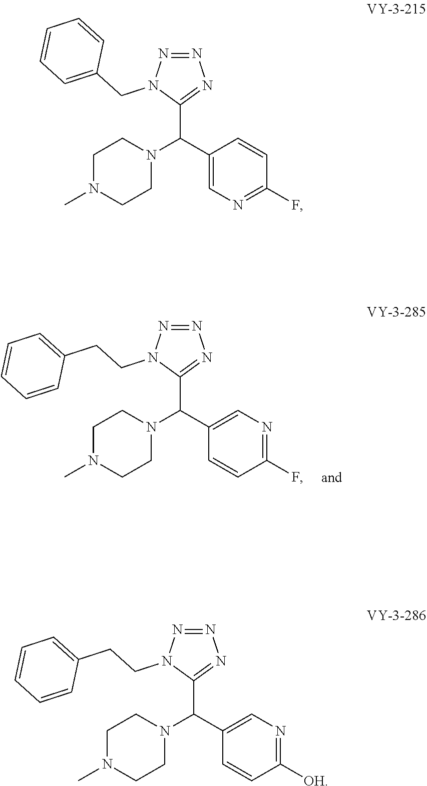 [SMILES: CN1CCN(C(c2ccc(F)nc2)c2nnnn2Cc2ccccc2)CC1 ; CN1CCN(C(c2ccc(F)nc2)c2nnnn2CCc2ccccc2)CC1 ; "and" ; CN1CCN(C(c2ccc(O)nc2)c2nnnn2CCc2ccccc2)CC1]

15. A method of treating, or ameliorating a disease or disorder that is caused, induced or characterized by abnormal reduction in glutamate transporter activity or abnormal increase in extracellular CNS glutamate concentration in a subject, the method comprising administering to the subject a therapeutically effective amount of a compound of claim 1.

16. The method of claim 15, wherein the disease or disorder is at least one selected from the group consisting of ischemia, seizure, traumatic brain injury, stroke, epilepsy, schizophrenia, and neurodegenerative diseases or disorders.

17. The method of claim 15, wherein the disease or disorder is at least one selected from the group consisting of ischemia, seizure, traumatic brain injury, stroke, epilepsy, schizophrenia, Parkinson's disease, Alzheimer's disease, Huntington's disease, amyotrophic lateral sclerosis (ALS), and ALS-parkinsonism dementia complex, HIV-associated neurocognitive disorder (HAND), neuropathic pain, mental health disorders and drug abuse/relapse.

18. The method of claim 15, wherein at least one of the following applies:
administration of the compound activates, stimulates, or upregulates the activity of a glutamate transporter in the subject, optionally wherein the transporter comprises EAAT2;
administration of the compound regulates extracellular glutamate concentrations in the subject;
administration of the compound increases, induces, or upregulates removal of glutamate from the neuronal synaptic cleft into neuroglia and neurons of the subject;
administration of the compound inhibits glutamate transport in the subject;
the compound is administered to the subject as part of a pharmaceutical composition;
the subject is further administered at least one additional therapeutic agent;
the subject is a mammal;
the subject is human.

19-26. (canceled)

27. The method of claim 15, wherein the compound is selected from the group consisting of 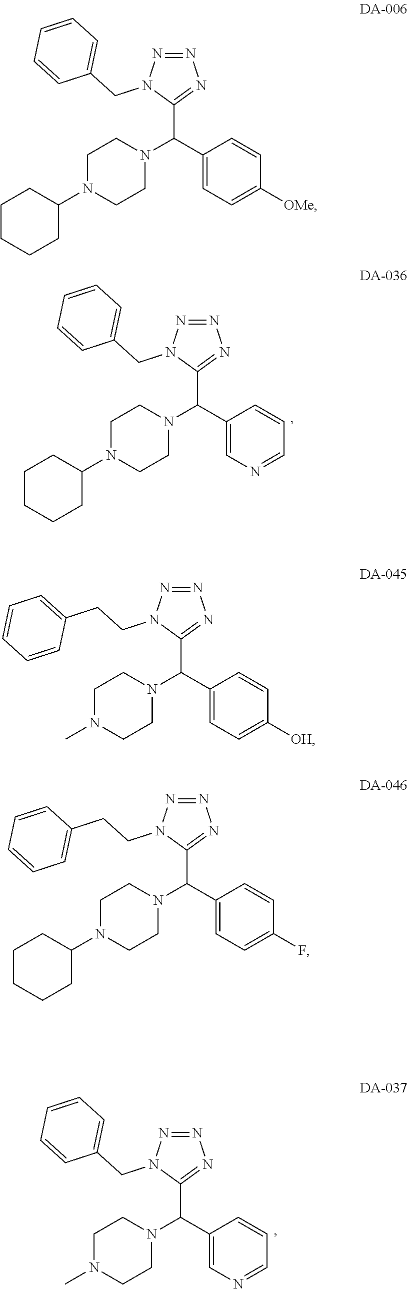 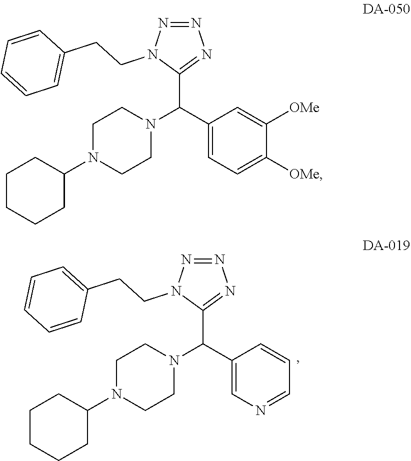 [SMILES: COc1ccc(C(c2nnnn2CCc2ccccc2)N2CCN(C3CCCCC3)CC2)cc1OC ; c1ccc(CCn2nnnc2C(c2cccnc2)N2CCN(C3CCCCC3)CC2)cc1 ; ","] 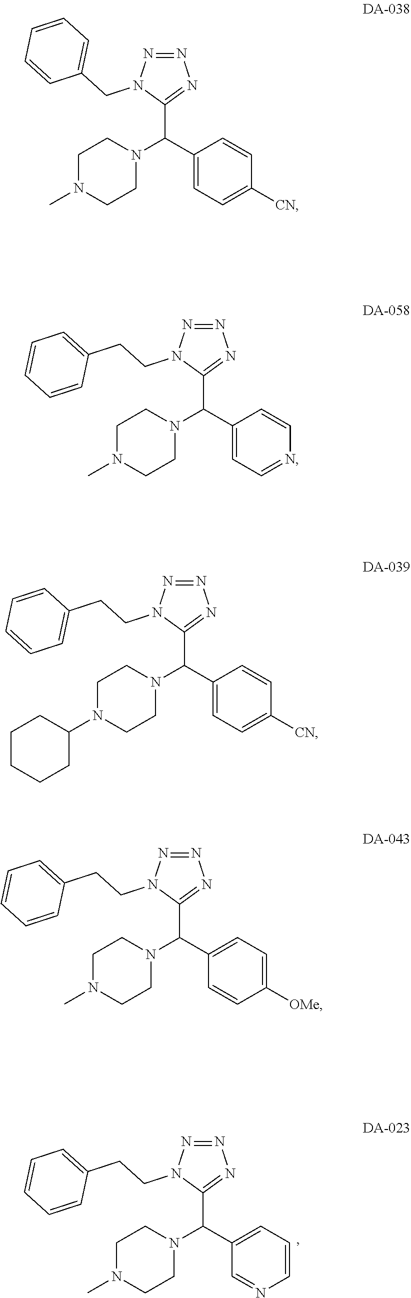 [SMILES: CN1CCN(C(c2ccc(C#N)cc2)c2nnnn2Cc2ccccc2)CC1 ; CN1CCN(C(c2ccncc2)c2nnnn2CCc2ccccc2)CC1 ; N#Cc1ccc(C(c2nnnn2CCc2ccccc2)N2CCN(C3CCCCC3)CC2)cc1 ; COc1ccc(C(c2nnnn2CCc2ccccc2)N2CCN(C)CC2)cc1 ; CN1CCN(C(c2cccnc2)c2nnnn2CCc2ccccc2)CC1 ; ","] 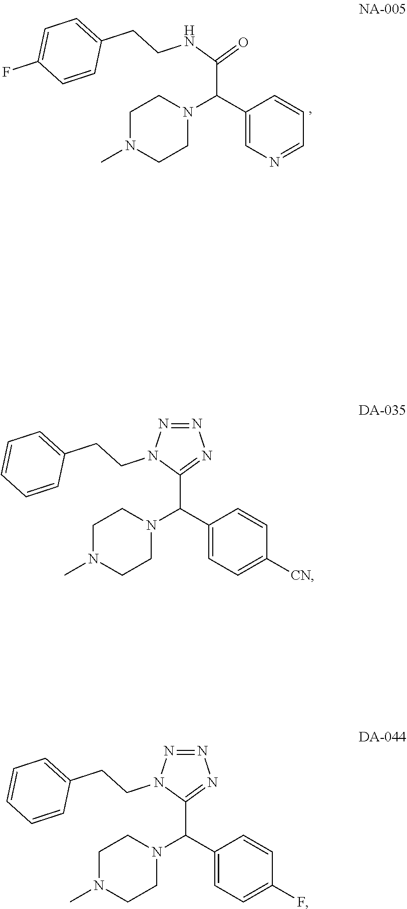 [SMILES: CN1CCN(C(C(=O)NCCc2ccc(F)cc2)c2cccnc2)CC1 ; "," ; CN1CCN(C(c2ccc(C#N)cc2)c2nnnn2CCc2ccccc2)CC1 ; CN1CCN(C(c2ccc(F)cc2)c2nnnn2CCc2ccccc2)CC1] 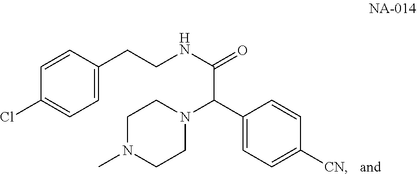 [SMILES: CN1CCN(C(C(=O)NCCc2ccc(Cl)cc2)c2ccc(C#N)cc2)CC1 ; "and"] 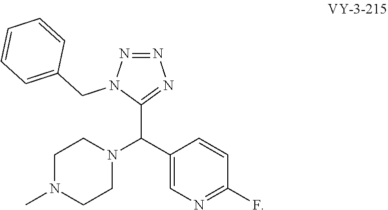 [SMILES: CN1CCN(C(c2ccc(F)nc2)c2nnnn2Cc2ccccc2)CC1]
